(19) United States
(12) Patent Application Publication
(10) Pub. No.: US 2003/0166509 A1
(43) Pub. Date: Sep. 4, 2003

(54) Compositions for sustained action product delivery and methods of use thereof

(75) Inventors: David A. Edwards, Boston, MA (US); Richard P. Batycky, Newton, MA (US); Jennifer L. Schmitke, Boston, MA (US); Nicolas Tsapis, Cambridge, MA (US); David A. Weitz, Bolton, MA (US); Jeffrey S. Hrkach, Cambridge, MA (US)

Correspondence Address: CAROLYN S. ELMORE, ESQ., ELMORE CRAIG, P. C., 209 MAIN STREET, NO. CHELMSFORD, MA, 01863, US

(73) Assignee: Advanced Inhalation Research, Inc., Cambridge, MA

(21) Appl. No.: 10/300,070

(22) Filed: Nov. 20, 2002

Related U.S. Application Data
(60) Provisional application No. 60/365,660, filed on Mar. 18, 2002.
(60) Provisional application No. 60/331,707, filed on Nov. 20, 2001.

(51) Int. Cl. A61K 38/28; A61L 9/04; A61K 9/14; A61K 31/56; A61K 31/4965; A61K 31/496; A61K 31/137

(52) U.S. Cl. 514/3; 424/46; 514/182; 514/252.13; 514/255.06; 514/651

(57) ABSTRACT

The present invention features pharmaceutical compositions comprising nanoparticles containing a sustained release bioactive agent, method of making such compositions, and method of therapy using such compositions.

### RELATED APPLICATIONS

This application claims the benefit of U.S. Provisional Application No. 60/365,660, filed Mar. 18, 2002 and U.S. Provisional Application No. 60/331,707 filed Nov. 20, 2001. The entire teachings of the above applications are incorporated herein by reference.

### BACKGROUND OF THE INVENTION

Product delivery, e.g., delivery of pharmaceutical or nutriceutical agents, often involves a delivery system which must be designed to satisfy multiple requirements. For example, a drug delivery system, such as a drug particle, ideally satisfies two distinct needs: it delivers the drug to the target site, or organ, and it releases the drug at the appropriate level and rate for pharmacodynamic action. Often these various needs require different attributes of the delivery system.

For example, inhaled particles deposit in the lungs if they possess a size range of approximately 1-5 microns (aerodynamic size). This makes such particles ideal for delivery of drugs to the lungs. On the other hand, the lungs clear such particles fairly rapidly after delivery. This means that inhaled drugs for sustained action are hampered by clearance of particles that optimally deposit in the lungs.

One way to solve this problem is to create large porous particles that can slow clearance, particularly in the alveolar region of the lungs where phagocytosis constitutes a primary form of clearance. This does not however solve the problem of delivery of particles to the respiratory tract, where mucociliary clearance effectively removes even large particles quite rapidly.

### SUMMARY OF THE INVENTION

We have found a solution to the problem of an effective delivery agent, e.g., for the lung and respiratory tract, and particularly, a kind of particle that can be useful for sustained release, and other kinds of delivery of bioactive agents, e.g., drugs and of nutraceutical agents, e.g., vitamins, minerals and food supplements. This particle is created as a spray dried particle with a size greater than a micron, containing small nanoparticles (e.g., 25 nanometers in size or larger, up to about 1 micron; also referred to herein as NPs), at mass fractions (per spray dried particle) of up to 100%, e.g., 100%, 95%, 90%, 80%, 75%, 60%, 50%, 30%, 25%, 10% and 5% that have agglomerated. The particles have the advantage of being easily delivered to a site in the body, for example, to the lungs by inhalation, and yet once they deposit, they can dissolve leaving behind primary nanoparticles that can escape clearance from the body. “Ultrafine” particles (nanoparticles) have been shown to potentially escape clearance and remain for long periods in the lungs (Chen et al., Journal of Colloid and Interface Science 190:118-133, 1997). Therefore such nanoparticles can deliver drugs more effectively or for longer periods of time.

Such particles can also be utilized in systems for other types of delivery, e.g., for oral delivery, particularly with sustained release. In oral delivery systems, the particles can be formulated to release the nanoparticles to a desired area of the gastrointestinal system. Such oral delivery systems can not only readily deliver bioactive agents, e.g., drugs and nutraceutical agents, e.g., vitamins, minerals and food supplements, but can also provide sustained delivery of those agents more easily than many other types of systems.

Accordingly, in one aspect, the invention features a pharmaceutical composition comprising spray dried particles, said particles comprising sustained action nanoparticles, said nanoparticles comprising a bioactive agent and having a geometric diameter of about 1 micron or less.

In another aspect, the invention features a method of treating a condition in a patient, comprising administering to said patient a pharmaceutical composition comprising spray dried particles, said particles comprising sustained action nanoparticles, said nanoparticles comprising a bioactive agent and having a geometric diameter of about 1 micron or less.

In another aspect, the invention features a method of making spray dried particles comprising sustained action nanoparticles, said nanoparticles comprising a bioactive agent and having a geometric diameter of about 1 micron or less, said method comprising the step of spray drying a solution comprising said nanoparticles under conditions that form spray dried particles.

In another aspect, the invention features a composition comprising spray dried particles, said particles comprising sustained action nanoparticles, said nanoparticles comprising a nutraceutical agent and having a geometric diameter of about 1 micron or less.

In another aspect, the invention features a method of treating a nutritional condition, e.g., a deficiency, in a patient comprising the step of administering to said patient a composition comprising spray dried particles, said particles comprising sustained action nanoparticles, said nanoparticles comprising a nutraceutical agent and having a geometric diameter of about 1 micron or less.

In another aspect, the invention features a method of making spray dried particles comprising sustained action nanoparticles, said nanoparticles comprising a bioactive agent and having a geometric diameter of about 1 micron or less, said method comprising the step of spray drying a solution comprising said nanoparticles under conditions that form spray dried particles. The particles of the present invention are made by forming nanoparticles (polymeric or nonpolymeric) with a clear size range and particle integrity. These nanoparticles contain one or more bioactive agents within them. The nanoparticles are dispersed in a solvent that contains other solutes useful for particle formation. The solution is spray dried, and the resulting particles are larger than a micron, porous, with excellent flow and aerodynamic properties. Such spray dried particles can be redissolved in solution, for example, physiologic fluids within the body to recover the original nanoparticles. The particles can be used to deliver various products, e.g., pharmaceutical and nutriceutical products, using various delivery modalities. In one embodiment, the particles are used as a pharmaceutical composition for pulmonary delivery. In particular, the particles can be designed to be deep lung depositing particles for the delivery of clearance resistant bioactive agent-containing nanoparticles that have size and composition characteristics that permit delivery of sustained release bioactive agents to difficult to reach areas of the pulmonary system. In one embodiment, the pharmaceutical composition is a therapeutic, diagnostic, or prophylactic composition.

### BRIEF DESCRIPTION OF THE DRAWINGS

FIG. 1 is a graph showing the variation of the mass median aerodynamic diameter (“MMAD”) and the geometric diameter of the dipalmitoyl phophatidylcholine-dimyristoyl phosphalidylethanolamine-lactose (“DPPC-DMPE-lactose”) solution spray dried according to a first set of spray drying conditions (“SD1”), described herein, using different concentrations of carboxylate modified latex (“CML”) polystyrene beads (170 nm in diameter).

FIG. 2A is a scanning electron microscopic (“SEM”) image of particles spray dried with conditions SD1 from the DPPC-DMPE-lactose solution containing no beads.

FIG. 2B is an SEM image of particles spray dried with conditions SD1 from the DPPC-DMPE-lactose solution containing 8.5% beads.

FIG. 2C is an SEM image of particles spray dried with conditions SD1 from the DPPC-DMPE-lactose solution containing 75% beads.

FIG. 2D is an SEM image of particles spray dried with conditions SD1 from the DPPC-DMPE-lactose solution containing 75% beads, viewed at a higher magnification.

FIG. 3A is a graph showing the variation of the MMAD of the DPPC-DMPE-lactose solution spray dried according to conditions SD1, with different concentrations of CML polystyrene beads (25 nm and 1 μm in diameter).

FIG. 3B is a graph showing the variation of the geometric diameter of the DPPC-DMPE-lactose solution spray dried according to conditions SD1, with different concentrations of CML polystyrene beads (25 nm and 1 μm in diameter).

FIG. 4 is a graph of the variation of the MMAD and the geometric diameter of the DPPC-DMPE-lactose solution spray dried according to a second set of spray drying conditions (“SD2”), with different polystyrene bead concentration (170 nm in diameter).

FIG. 5A is an SEM image of particles spray dried according to conditions SD2 from the DPPC-DMPE-lactose solution containing no beads.

FIG. 5B is an SEM image of particles spray dried according to conditions SD2 from the DPPC-DMPE-lactose solution containing 35% beads.

FIG. 5C is an SEM image of particles spray dried according to conditions SD2 from the DPPC-DMPE-lactose solution containing 82% beads.

FIG. 6A is an SEM image of particles spray dried from the DPPC-DMPE-lactose solution containing 88% colloidal silica (w/w).

FIG. 6B is an SEM image of particles spray dried from the DPPC-DMPE-lactose solution containing 88% colloidal silica (w/w) viewed at a higher magnification.

FIG. 7 is a graph of the variation of the MMAD and the geometric diameter of the DPPC-DMPE-lactose with different concentrations of colloidal silica.

FIG. 8A is an SEM image of spray dried particles made of BSA containing 78% CML polystyrene beads(w/w).

FIG. 8B is an SEM image of spray dried particles made of insulin containing 80.2% CML polystyrene beads(w/w).

FIG. 9A is an SEM image of laboratory-designed polystyrene beads generated as described herein.

FIG. 9B is an SEM image of laboratory designed polystyrene beads generated as described herein.

FIG. 10 is a graph of the variation of the reverse of the characteristic time (τ) of the intensity autocorrelation function with the wave vector (q) to the square. The slope of the straight line which gives the best fit gives the diffusion coefficient of the laboratory-designed polystyrene beads generated as described herein.

FIG. 11A is an SEM image of spray dried particles containing laboratory-designed polystyrene beads generated as described herein.

FIG. 11B is an SEM image of spray dried particles containing laboratory-designed polystyrene beads generated as described herein.

FIG. 11C is an SEM image of spray dried particles containing laboratory-designed polystyrene beads generated as described herein.

FIG. 11D is an SEM image of spray dried particles containing laboratory-designed polystyrene beads generated as described herein.

FIG. 12A is an SEM image of a DPPC-DMPE-lactose powder containing laboratory-designed polystyrene beads, generated as described herein, after dissolution in ethanol.

FIG. 12B is an SEM image of a DPPC-DMPE-lactose powder containing laboratory-designed polystyrene beads, generated as described herein, after dissolution in a mixture of ethanol/water (70/30 (v/v)).

FIG. 13A is a graph of the time evolution of UV spectra of laboratory-designed dried beads containing estradiol in ethanol.

FIG. 13B is a graph of the OD of the 274 nm peak of the graph shown in FIG. 13A plotted versus time.

FIG. 14 is a graph of the variation of estradiol concentration in rat plasma after subcutaneous injection of estradiol loaded laboratory-designed beads or plain estradiol loaded powder at time T=0.

FIG. 15 is a schematic representation of the generation of sprayed dried particles with characteristics that provide for deposition to the alveolar region of the lungs, and the use of spray dried particles containing nanoparticles and lipids to form such particles.

FIG. 16 is a schematic representation of various characteristic of spray dried particles containing nanoparticles, as described herein, including scanned images of the particles, a graph showing the effect of increasing the concentration of the nanoparticles in the particles on the geometric diameter, and a schematic representation of the particles that are formed using the methods described herein.

FIG. 17 shows SEMs of particles of the present invention containing lipids+colloidal silica, bovine serum albumin+polystyrene beads, or micelles of diblock polymers, as well as a list of some of the characteristics of the particles of the present invention.

FIG. 18A is an SEM image of a typical hollow sphere observed from the spray drying of a solution of polystyrene nanoparticles (170 nm). The lower image is a zoom on the particle surface.

FIG. 18B is an SEM image of a zoom on the particle surface of a typical hollow sphere observed from the spray drying of a solution of polystyrene nanoparticles (170 nm).

FIG. 19A is an SEM image of a typical hollow sphere observed from the spray drying of a solution of polystyrene nanoparticles (25 nm). The scale bar is 10 μm.

FIG. 19B is an SEM image of a typical hollow sphere observed from the spray drying of a solution of polystyrene nanoparticles (25 nm). The scale bar is 2 μm.

FIG. 20A is an SEM image of a typical hollow sphere observed from the spray drying of a solution of lactose and polystyrene nanoparticles (170 nm 70% of total solid contents in weight). The scale bar is 10 μm.

FIG. 20B is an SEM image of a typical hollow sphere observed from the spray drying of a solution of lactose and polystyrene nanoparticles (170 nm 70% of total solid contents in weight). The scale bar is 2 μm.

FIG. 21A is an SEM image of a typical hydroxypropylcellulose spray-dried particle without nanoparticles. The scale bar represents 2 μm.

FIG. 21B is an SEM image of a typical hydroxypropylcellulose spray-dried particle without with nanoparticles. (top right). Scale bar represents 20 μm.

FIG. 21C is an SEM image of a zoom on the particle surface of a typical hydroxypropylcellulose spray-dried particle with nanoparticles. The scale bar represents 2 μm.

FIG. 22A is an SEM image of the particles resulting from the spray-drying of a solution of Rifampicin, DPPC, DMPE and lactose in ethanol/water (70/30 v/v). The Rifampicin concentration was 40% by weight of solid contents in the solution. The scale bar represents 5 μm.

FIG. 22B is an SEM image of the particles resulting from the spray-drying of a solution of Rifampicin, DPPC, DMPE and lactose in ethanol/water (70/30 v/v). The Rifampicin concentration was 40% by weight of solid contents in the solution. The scale bar represents 2 μm.

FIG. 23A is an SEM image of the particles resulting from the spray-drying of a solution of Rifampicin, DPPC, DMPE and lactose in ethanol/water (70/30 v/v). The Rifampicin concentration was 40% by weight of solid contents in the solution. The scale bar represents 2 μm.

FIG. 23B is an SEM image of the particles resulting from the spray-drying of a solution of Rifampicin, DPPC, DMPE and lactose in ethanol/water (70/30 v/v). The Rifampicin concentration was 40% by weight of solid contents in the solution. The scale bar represents 500 nm.

FIG. 23C is an SEM image of the particles resulting from the spray-drying of a solution of Rifampicin, DPPC, DMPE and lactose in ethanol/water (70/30 v/v). The Rifampicin concentration was 20% by weight of solid contents in the solution. The scale bar represents 1 μm.

FIG. 23D is an SEM image of the particles resulting from the spray-drying of a solution of Rifampicin, DPPC, DMPE and lactose in ethanol/water (70/30 v/v). The Rifampicin concentration was 60% by weight of solid contents in the solution. The scale bar represents 2 μm.

FIG. 24A is an SEM image of the particles resulting from the spray-drying of a solution of Rifampicin (1 g/L) alone in a mixture of ethanol/water (70/30 v/v) (with 1% chloroform)

FIG. 24B is an SEM image of the particles resulting from the spray-drying of a solution of Rifampicin (1 g/L) in “pure” ethanol (with 1% chloroform).

FIG. 24C is an SEM image of the particles resulting from the spray-drying of a solution of Rifampicin (1 g/L) with lipids (60/40 w/w) in “pure” ethanol (with 1% chloroform).

FIG. 25A is an SEM image of spray dried particles from Rifampicin-DPPC (60/40 w/w) solutions containing salts (sodium citrate/calcium chloride) or not containing salts.

FIG. 25B is an SEM image of spray dried particles from Rifampicin-DPPC (60/40 w/w) solutions containing salts (sodium citrate/calcium chloride).

FIG. 25C is an SEM image of spray dried particles from Rifampicin-DPPC (60/40 w/w) solutions containing salts (sodium citrate/calcium chloride).

FIG. 25D is an SEM image of spray dried particles from Rifampicin-DPPC (60/40 w/w) solutions not containing salts.

### DETAILED DESCRIPTION OF THE INVENTION

The features and other details of the invention, either as steps of the invention or as combination of parts of the invention, will now be more particularly described with reference to the accompanying drawings and pointed out in the claims. The drawings are not necessarily to scale, with emphasis instead being placed upon illustrating the principles of the invention. It will be understood that the particular embodiments of the invention are shown by way of illustration and not as limitations of the invention. The principle feature of this invention may be employed in various embodiments without departing from the scope of the invention.

Particle and Nanoparticle Formation

The particles of the present invention can be formed using spray drying techniques. In such techniques, a spray drying mixture, also referred to herein as “feed solution” or “feed mixture,” is formed to include nanoparticles comprising a bioactive agent and, optionally, one or more additives that are fed to a spray dryer.

Suitable organic solvents that can be present in the mixture to be spray dried include, but are not limited to, alcohols, for example, ethanol, methanol, propanol, isopropanol, butanols, and others. Other organic solvents include, but are not limited to, perfluorocarbons, dichloromethane, chloroform, ether, ethyl acetate, methyl tert-butyl ether and others. Another example of an organic solvent is acetone. Aqueous solvents that can be present in the feed mixture include water and buffered solutions. Both organic and aqueous solvents can be present in the spray-drying mixture fed to the spray dryer. In one embodiment, an ethanol water solvent is preferred with the ethanol:water ratio ranging from about 20:80 to about 90:10. The mixture can have an acidic or an alkaline pH. Optionally, a pH buffer can be included. Preferably, the pH can range from about 3 to about 10. In another embodiment, the pH ranges from about 1 to about 13.

The total amount of solvent or solvents employed in the mixture being spray dried generally is greater than about 97 weight percent. Preferably, the total amount of solvent or solvents employed in the mixture being spray dried generally is greater than about 99 weight percent The amount of solids (nanoparticles containing bioactive agent, additives, and other ingredients) present in the mixture being spray dried generally is less than about 3.0 weight percent. Preferably, the amount of solids in the mixture being spray dried ranges from about 0.05% to about 1.0% by weight.

The spray dried particles of the present invention comprise nanoparticles containing one or more bioactive agents. Nanoparticles can be produced according to methods known in the art, for example, emulsion polymerization in a continuous aqueous phase, emulsion polymerization in a continuous organic phase, milling, precipitation, sublimation, interfacial polycondensation, spray drying, hot melt microencapsulation, phase separation techniques (solvent removal and solvent evaporation), nanoprecipitation as described by A. L. Le Roy Boehm, R. Zerrouk and H. Fessi (J. Microencapsulation, 2000, 17: 195-205) and phase inversion techniques. Additional methods for producing are evaporated precipitation, as described by Chen et al. (International Journal of Pharmaceutics, 2002, 24, pp 3-14) and through the use of supercritical carbon dioxide as an anti-solvent (as described, for example, by J. -Y. Lee et al., Journal of Nanoparticle Research, 2002, 2, pp 53-59). Nanocapsules can be produced by the method of F. Dalencon, Y. Amjaud, C. Lafforgue, F. Derouin and H. Fessi (International Journal of Pharmaceutics ,1997, 153:127-130). U.S. Pat. Nos. 6,143,211, 6,117,454 and 5,962,566; Amnoury (J. Pharm. Sci., 1990, pp 763-767); Julienne et al., (Proceed. Intern. Symp. Control. Rel. Bioact. Mater., 1989, pp 77-78); Bazile et al. (Biomaterials 1992, pp 1093-1102); Gref et al. (Science 1994, 263, pp 1600-1603); Colloidal Drug Delivery Systems (edited by Jorg Kreuter, Marcel Dekker, Inc., New York, Basel, Hong Kong, pp 219-341); and International. Patent Application No. WO 00/27363, the entire teachings of each of which are hereby incorporated by reference, describe the manufacture of nanoparticles and incorporation of bioactive agents, for example, drugs, in the nanoparticles.

The nanoparticles of the present invention can be polymeric, and such polymeric nanoparticles can be biodegradable or nonbiodegradable. For example, polymers used to produce the nanoparticles include, but are not limited to polyamides, polyanhydrides, polystyrenes, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl acetate), poly vinyl chloride, ethylene vinyl acetate, polyamino acids (e.g., polyleucine), lactic acid, polylactic acid, glycolic acid, poly(ortho)esters, polyurethanes, poly(butic acid), poly(valeric acid), poly(caprolactone), poly(hydroxybutyrate), poly(lactide-co-glycolide) and poly(lactide-co-caprolactone), poly(lactide-co-glycolide), and copolymers and mixtures thereof, and natural polymers such as alginate and other polysaccharides including dextran and cellulose, collagen, including chemical derivatives thereof, albumin and other hydrophilic proteins, zein and other prolamines and hydrophobic proteins, and copolymers and mixtures thereof. Another polymer that can be used to produce the nanoparticles of the present invention is poly(alkylcyanoacrylate). In general, nanoparticles formed from biodegradable materials degrade either by enzymatic hydrolysis or exposure to water in vivo, by surface or bulk erosion. The foregoing materials may be used alone, as physical mixtures (blends), or as co-polymers.

The nanoparticles of the present inventions can alternatively be nonpolymeric. Examples of useful non-polymeric materials include, but are not limited to silica, sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈ -C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols; and combinations and mixtures thereof. In one embodiment, the nanoparticles are made of antibiotics.

Bioactive agents also are referred to herein as bioactive compounds, drugs or medicaments. Once the particles are delivered to the pulmonary region, they dissolve leaving behind the nanoparticles, which are small enough to escape clearance from the lung by the macrophage. The nanoparticles then provide sustained action delivery of the bioactive agent. The particles can also contain as an active agent one or more nutraceutical agents. As the term “nutraceutical agent” is used herein, it includes any compound that provides nutritional benefit. Nutraceutical agents include, but are not limited to, vitamins, minerals and other nutritional supplements. Nutraceuticals can be obtained from natural sources or can be synthesized. The term “sustained action”, as used herein, means that the period of time for which a bioactive agent released and made bioavailable from a nanoparticle containing a certain amount of bioactive agent is greater than the period of time for which the same bioactive agent, in the same amount and under the same conditions, but not contained in a nanoparticle is released and made bioavailable, for example, following direct administration of the bioactive agent. This can be assayed using standard methods, for example, by measuring serum levels of the bioactive agent or by measuring the amount of bioactive agent released into a solvent. A sustained release bioactive agent can be released, for example, three to five times slower from a nanoparticle, compared to the same bioactive agent not contained in a nanoparticle. Alternatively, the period of sustained release of a bioactive agent occurs over a period of at least one hour, for example, at least 12, 24, 36 or 48 hours. Preferably, the bioactive agent is delivered to a target site, for example, a tissue, organ or entire body in an effective amount. As used herein, the term “effective amount” means the amount needed to achieve the desired therapeutic or diagnostic effect or efficacy. The actual effective amounts of bioactive agent can vary according to the specific bioactive agent or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations, e.g., by means of an appropriate, conventional pharmacological protocol. In one embodiment, the bioactive agent is coated onto the nanoparticle.

Suitable bioactive agents include agents that can act locally, systemically or a combination thereof. The term “bioactive agent,” as used herein, is an agent, or its pharmaceutically acceptable salt, which when released in vivo, possesses the desired biological activity, for example therapeutic, diagnostic and/or prophylactic properties in vivo. Examples of bioactive agents include, but are not limited to, synthetic inorganic and organic compounds, proteins, peptides, polypeptides, DNA and RNA nucleic acid sequences or any combination or mimic thereof, having therapeutic, prophylactic or diagnostic activities. The agents to be incorporated can have a variety of biological activities, such as vasoactive agents, neuroactive agents, hormones, anticoagulants, immunomodulating agents, cytotoxic agents, prophylactic agents, antibiotics, antivirals, antisense, antigens, and antibodies. Another example of a biological activity of the bioactive agents is bacteriostatic activity. Compounds with a wide range of molecular weight can be used, for example, compounds with weights between 100 and 500,000 grams or more per mole.

Nutriceutical agents are also suitable for use as components of the particles and the nanoparticles.. Such agents include vitamins, minerals and nutritional supplements.

“Polypeptides,” as used herein, means any chain of more than two amino acids, regardless of post-translational modification such as glycosylation or phosphorylation. Examples of polypeptides include, but are not limited to, complete proteins, muteins and active fragments thereof, such as insulin, immunoglobulins, antibodies, cytokines (e.g., lymphokines, monokines, chemokines), interleukins, interferons (β-IFN, α-IFN and γ-IFN), erythropoietin, nucleases, tumor necrosis factor, colony stimulating factors, enzymes (e.g., superoxide dismutase, tissue plasminogen activator), tumor suppressors, blood proteins, hormones and hormone analogs (e.g., growth hormone, adrenocorticotropic hormone and luteinizing hormone releasing hormone (“LHRH”), vaccines, e.g., tumoral, bacterial and viral antigens, antigens, blood coagulation factors; growth factors; granulocyte colony-stimulating factor (“G-CSF”); polypeptides include protein inhibitors, protein antagonists, and protein agonists, calcitonin. “Nucleic acid” as used herein refers to DNA or RNA sequences of any length and include genes and antisense molecules which can, for instance, bind to complementary DNA to inhibit transcription, and ribozymes. Polysaccharides, such as heparin, can also be administered. Particularly useful bioactive agents are drugs for the treatment of asthma, for example, albuterol, drugs for the treatment of tuberculosis, for example, rifampin, ethambutol and pyrazinamide as well as drugs for the treatment of diabetes such as Humulin Lente® (Humulin L®; human insulin zinc suspension), Humulin R® (regular soluble insulin (RI)), Humulin Ultralente® (Humulin U®), and Humalog 100® (insulin lispro (IL)) from Eli Lilly Co. (Indianapolis, Ind.; 100 U/mL). Other examples of bioactive agents for use in the present invention include isoniacide, para-amino salicylic acid, cycloserine, streptomycin, kanamycin, and capreomycin. Rifampin is also known as Rifampicin.

Bioactive agents for local delivery within the lung, include such agents as those for the treatment of asthma, chronic obstructive pulmonary disease (COPD), emphysema, or cystic fibrosis. For example, genes for the treatment of diseases such as cystic fibrosis can be administered, as can beta agonists steroids, anticholinergics, and leukotriene modifers for asthma.

Other specific bioactive agents include estrone sulfate, albuterol sulfate, parathyroid hormone-related peptide, somatostatin, nicotine, clonidine, salicylate, cromolyn sodium, salmeterol, formeterol, L-dopa, Carbidopa or a combination thereof, gabapenatin, clorazepate, carbamazepine and diazepam.

The nanoparticles can include any of a variety of diagnostic agents to locally or systemically deliver the agents following administration to a patient. For example, imaging agents which include commercially available agents used in positron emission tomography (PET), computer assisted tomography (CAT), single photon emission computerized tomography, x-ray, fluoroscopy, and magnetic resonance imaging (MRI) can be employed.

Examples of suitable materials for use as contrast agents in MRI include the gadolinium chelates currently available, such as diethylene triamine pentacetic acid (DTPA) and gadopentotate dimeglumine, as well as iron, magnesium, manganese, copper and chromium.

Examples of materials useful for CAT and x-rays include iodine based materials for intravenous administration, such as ionic monomers typified by diatrizoate and iothalamate, and ionic dimers, for example, ioxagalte.

Diagnostic agents can be detected using standard techniques available in the art and commercially available equipment. In addition, the nanoparticles of the present invention can contain one or more of the following bioactive materials which can be used to detect an analyte: an antigen, an antibody (monoclonal or polyclonal), a receptor, a hapten, an enzyme, a protein, a polypeptide, a nucleic acid (e.g., DNA or RNA) a drug, a hormone, or a polymer, or combinations thereof. If desired, the diagnostic can be detectably labeled for easier diagnostic use. Examples of such labels include, but are not limited to various enzymes, prosthetic groups, fluorescent materials, luminescent materials, bioluminescent materials, and radioactive materials. Examples of suitable enzymes include horseradish peroxidase, alkaline phosphatase, β-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; examples of bioluminescent materials include luciferase, luciferin, and aequorin, and examples of suitable radioactive material include ¹²⁵I, ¹³¹I, ³⁵S, and ³H.

The nanoparticles can contain from about 0.01% (w/w) to about 100% (w/w) e.g., 0.01%, 0.05%, 0.10%, 0.25%, 0.50%, 1.00%, 2.00%, 5.00%, 10.00%, 20.00%, 30.00%, 40.00%, 50.00%, 60.00%, 75.00%, 80.00%, 85.00%, 90.00%, 95.00%, 99.00% or more, of bioactive agent (dry weight of composition). The amount of bioactive agent used will vary depending upon the desired effect, the planned release levels, and the time span over which the bioactive agent will be released. The amount of bioactive agent present in the nanoparticles in the liquid feed generally ranges between about 0.1% weight and about 100% weight, preferably between about 1.0% weight and about 100% weight. Combinations of bioactive agents also can be employed.

Intact (preformed) nanoparticle can be added to the solution(s) to be spray dried. Alternatively, reagents capable of forming nanoparticles during the mixing and/or spray drying process can be added to the solutions to be spray dried. Such reagents include those described in Example 15 herein. In one embodiment, the reagents are capable of forming nanoparticles under spray drying conditions described herein. In another embodiment, the reagents are capable of forming nanoparticles under spray drying conditions described in Example 15.

In addition to the spray dried particles of the present invention comprising bioactive agent-containing nanoparticles, the spray dried particles can include one or more additional components (additives). As used herein, an additive is any substance that is added to another substance to produce a desired effect in, or in combination with, the primary substance. In a preferred embodiment, liquid to be spray dried optionally includes one or more phospholipids, such as, for example, a phosphatidylcholine, phosphatidylethanolamine, phosphatidylglycerol, phosphatidylserine, phosphatidylinositol or a combination thereof. In one embodiment, the phospholipids are endogenous to the lung. Specific examples of phospholipids are shown in Table 1. Combinations of phospholipids can also be employed.

TABLE 1

| | |
|---|---|
| Dilaurylolyphosphatidylcholine (C12;0) | DLPC |
| Dimyristoylphosphatidylcholine (C14;0) | DMPC |
| Dipalmitoylphosphatidylcholine (C16:0) | DPPC |
| Distearoylphosphatidylcholine (C18:0) | DSPC |
| Dioleoylphosphatidylcholine (C18:1) | DOPC |
| Dilaurylolylphosphatidylglycerol | DLPG |
| Dimyristoylphosphatidylglycerol | DMPG |
| Dipalmitoylphosphatidylglycerol | DPPG |
| Distearoylphosphatidylglycerol | DSPG |
| Dioleoylphosphatidylglycerol | DOPG |
| Dimyristoyl phosphatidic acid | DMPA |
| Dimyristoyl phosphatidic acid | DMPA |
| Dipalmitoyl phosphatidic acid | DPPA |
| Dipalmitoyl phosphatidic acid | DPPA |
| Dimyristoyl phosphatidylethanolamine | DMPE |
| Dipalmitoyl phosphatidylethanolamine | DPPE |
| Dimyristoyl phosphatidylserine | DMPS |
| Dipalmitoyl phosphatidylserine | DPPS |
| Dipalmitoyl sphingomyelin | DPSP |
| Distearoyl sphingomyelin | DSSP |

Charged phospholipids also can be employed to generate particles that contain nanoparticles comprising bioactive agents. Examples of charged phospholipids are described in U.S. patent application entitled “Particles for Inhalation Having Sustained Release Properties,” Ser. No. 09/752,106 filed on Dec. 29, 2000, and in U.S. patent application Ser. No. , 09/752,109 entitled “Particles for Inhalation Having Sustained Release Properties”, filed on Dec. 29, 2000; the entire contents of both are incorporated herein by reference.

The phospholipid can be present in the particles in an amount ranging from about 5 weight percent (%) to about 95 weight %. Preferably, it can be present in the particles in an amount ranging from about 20 weight % to about 80 weight %.

In one embodiment of the invention, the particles optionally also include a bioactive agent, for example, a therapeutic, prophylactic or diagnostic agent as an additive. This bioactive agent may be the same or different from the bioactive agent contained in the nanoparticles. The amount of bioactive agent used will vary depending upon the desired effect, the planned release levels, and the time span over which the bioactive agent will be released. A preferred range of bioactive agent loading in alternative compositions is between about 0.1% (w/w) to about 100% (w/w) bioactive agent, e.g., 0.01%, 0.05%, 0.10%, 0.25%, 0.50%, 1.00%, 2.00%, 5.00%, 10.00%, 20.00%, 30.00%, 40.00%, 50.00%, 60.00%, 75.00%, 80.00%, 85.00%, 90.00%, 95.00%, 99.00% or more. Combinations of bioactive agents also can be employed.

In another embodiment of the invention, the additive is an excipient. As used herein, an “excipient” means a compound that is added to a pharmaceutical formulation in order to confer a suitable consistency. For example, the particles can include a surfactant. As used herein, the term “surfactant” refers to any agent which preferentially absorbs to an interface between two immiscible phases, such as the interface between water and an organic polymer solution, a water/air interface, a water/oil interface, a water/organic solvent interface or an organic solvent/air interface. Surfactants generally possess a hydrophilic moiety and a lipophilic moiety, such that, upon absorbing to microparticles, they tend to present moieties to the external environment that do not attract similarly-coated particles, thus reducing particle agglomeration. Surfactants may also promote absorption of a therapeutic or diagnostic agent and increase bioavailability of the agent.

In addition to lung surfactants, such as, for example, the phospholipids discussed previously, suitable surfactants include but are not limited to phospholipids, polypeptides, polysaccharides, polyanhydrides, amino acids, polymers, proteins, surfactants, cholesterol, fatty acids, fatty acid esters, sugars, hexadecanol; fatty alcohols such as polyethylene glycol (PEG); polyoxyethylene-9-lauryl ether; a surface active fatty acid, such as palmitic acid or oleic acid; glycocholate; surfactin; a poloxamer; a sorbitan fatty acid ester such as sorbitan trioleate (Span 85), Tween 80 (Polyoxyethylene Sorbitan Monooleate); tyloxapol, polyvinyl alcohol (PVA), and combinations thereof.

The surfactant can be present in the liquid feed in an amount ranging from about 0.01 weight % to about 5 weight %. Preferably, it can be present in the particles in an amount ranging from about 0.1 weight % to about 1.0 weight %.

Methods of preparing and administering particles including surfactants, and, in particular phospholipids, are disclosed in U.S. Pat. No 5,855,913, issued on Jan. 5, 1999 to Hanes et al. and in U.S. Pat. No. 5,985,309, issued on Nov. 16, 1999 to Edwards et al. The teachings of both are incorporated herein by reference in their entirety.

The particles can further comprise a carboxylic acid which is distinct from the agent and lipid, in particular a phospholipid. In one embodiment, the carboxylic acid includes at least two carboxyl groups. Carboxylic acids, include the salts thereof as well as combinations of two or more carboxylic acids and/or salts thereof. In a preferred embodiment, the carboxylic acid is a hydrophilic carboxylic acid or salt thereof. Suitable carboxylic acids include but are not limited to hydroxydicarboxylic acids, hydroxytricarboxilic acids and the like. Citric acid and citrates, such as, for example sodium citrate, are preferred. Combinations or mixtures of carboxylic acids and/or their salts also can be employed.

The carboxylic acid can be present in the particles in an amount ranging from about 0.1 % to about 80% by weight. Preferably, the carboxylic acid can be present in the particles in an amount of about 10% to about 20% by weight.

The particles suitable for use in the invention can further comprise an amino acid. In a preferred embodiment the amino acid is hydrophobic. Suitable naturally occurring hydrophobic amino acids, include but are not limited to, leucine, isoleucine, alanine, valine, phenylalanine, glycine and tryptophan. Combinations of hydrophobic amino acids can also be employed. Suitable non-naturally occurring amino acids include, for example, beta-amino acids. Both D, L configurations and racemic mixtures of hydrophobic amino acids can be employed. Suitable hydrophobic amino acids can also include amino acid derivatives or analogs. As used herein, an amino acid analog includes the D or L configuration of an amino acid having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C1-C8 hydrocarbons which are completely saturated, which contain one or two heteroatoms such as nitrogen, oxygen or sulfur and/or which contain one or more units of unsaturation. Aromatic or aryl groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

A number of the suitable amino acids, amino acids analogs and salts thereof can be obtained commercially. Others can be synthesized by methods known in the art. Synthetic techniques are described, for example, in Green and Wuts, “Protecting Groups in Organic Synthesis”, John Wiley and Sons, Chapters 5 and 7, 1991.

Hydrophobicity is generally defined with respect to the partition of an amino acid between a nonpolar solvent and water. Hydrophobic amino acids are those acids which show a preference for the nonpolar solvent. Relative hydrophobicity of amino acids can be expressed on a hydrophobicity scale on which glycine has the value 0.5. On such a scale, amino acids which have a preference for water have values below 0.5 and those that have a preference for nonpolar solvents have a value above 0.5. As used herein, the term “hydrophobic amino acid” refers to an amino acid that, on the hydrophobicity scale has a value greater or equal to 0.5, in other words, has a tendency to partition in the nonpolar acid which is at least equal to that of glycine.

Examples of amino acids which can be employed include, but are not limited to: glycine, proline, alanine, cysteine, methionine, valine, leucine, tyrosine, isoleucine, phenylalanine, tryptophan. Preferred hydrophobic amino acids include leucine, isoleucine, alanine, valine, phenylalanine, glycine and tryptophan. Combinations of hydrophobic amino acids can also be employed. Furthermore, combinations of hydrophobic and hydrophilic (preferentially partitioning in water) amino acids, where the overall combination is hydrophobic, can also be employed. Combinations of one or more amino acids can also be employed.

The amino acid can be present in the particles of the invention in an amount from about 0% to about 60 weight %. Preferably, the amino acid can be present in the particles in an amount ranging from about 5 weight % to about 30 weight %. The salt of a hydrophobic amino acid can be present in the particles of the invention in an amount of from about 0% to about 60 weight %. Preferably, the amino acid salt is present in the particles in an amount ranging from about 5 weight % to about 30 weight %. Methods of forming and delivering particles which include an amino acid are described in U.S. patent application Ser. No. 09/382,959, filed on Aug. 25, 1999, entitled Use of Simple Amino Acids to Form Porous Particles During Spray Drying, and U.S. patent application Ser. No. 09/644,320, filed on Aug. 23, 2000, entitled Use of Simple Amino Acids to Form Porous Particles, the entire teachings of which are incorporated herein by reference.

It is understood that when the particles includes a carboxylic acid, a multivalent salt, an amino acid, a surfactant or any combination thereof, that interaction between these components of the particle and the charged lipid can occur.

In a further embodiment, the particles of the present invention can also include other additives, for example, buffer salts, dextran, polysaccharides, lactose, trehalose, cyclodextrins, proteins, peptides, polypeptides, fatty acids, fatty acid esters, inorganic compounds, and phosphates.

In one embodiment of the invention, the particles can further comprise polymers. The use of polymers can further prolong release. Biocompatible or biodegradable polymers are preferred. Such polymers are described, for example, in U.S. Pat. No. 5,874,064, issued on Feb. 23, 1999 to Edwards et al., the teachings of which are incorporated herein by reference in their entirety. Additional polymers that can be used to form the particles of the present invention include those described above for the formation of nanoparticles.

Any of the above described additives can also be used to make the nanoparticles of the present invention.

It will be understood that the choice of materials contained in the particle and nanoparticle, including bioactive agents and additives will be dictated by the desired pharmaceutical effect of the particle, and can be chosen, without limitation and difficulty, by one of skill in the art.

The particles of the instant invention, are a respirable pharmaceutical composition suitable for pulmonary delivery. As used herein, the term “respirable” means suitable for being breathed, or adapted for respiration. “Pulmonary delivery,” as that term is used herein, means delivery to the respiratory tract. The “respiratory tract,” as the term is used herein, encompasses the upper airways, including the oropharynx and larynx, followed by the lower airways, which include the trachea followed by bifurcations into the bronchi and bronchioli (e.g., terminal and respiratory). The upper and lower airways are termed the conducting airways. The terminal bronchioli then divide into respiratory bronchioli which then lead to the ultimate respiratory zone, namely, the alveoli, or deep lung. The deep lung, or alveoli, are typically the desired the target of inhaled therapeutic formulations for systemic bioactive agent delivery.

The spray dryer used to form the particle of the present invention can employ a centrifugal atomization assembly, which includes a rotating disk or wheel to break the fluid into droplets, for example, a 24 vaned atomizer or a 4 vaned atomizer. The rotating disk typically operates within the range from about 1,000 to about 55,000 rotations per minute (rpm).

Alternatively, hydraulic pressure nozzle atomization, two fluid pneumatic atomization, sonic atomization or other atomizing techniques, as known in the art, also can be employed. Commercially available spray dryers from suppliers such as Niro, APV Systems, Denmark, (e.g., the APV Anhydro Model) and Swenson, Harvey, Ill., as well as scaled-up spray dryers suitable for industrial capacity production lines can be employed, to generate the particles as described herein. Commercially available spray dryers generally have water evaporation capacities ranging from about 1 to about 120 kg/hr. For example, a Niro Mobile Minor™ spray dryer has a water evaporation capacity of about 7 kg/hr. The spray driers have a 2 fluid external mixing nozzle, or a 2 fluid internal mixing nozzle (e.g., a NIRO Atomizer Portable spray dryer).

Suitable spray-drying techniques are described, for example, by K. Masters in “Spray Drying Handbook,” John Wiley & Sons, New York, 1984. Generally, during spray-drying, heat from a hot gas such as heated air or nitrogen is used to evaporate the solvent from droplets formed by atomizing a continuous liquid feed. Other spray-drying techniques are well known to those skilled in the art. In a preferred embodiment, a rotary atomizer is employed. An example of a suitable spray dryer using rotary atomization includes the Mobile Minor™ spray dryer, manufactured by Niro, Denmark. The hot gas can be, for example, air, nitrogen or argon.

Preferably, the particles of the invention are obtained by spray drying using an inlet temperature between about 90° C. and about 400° C. and an outlet temperature between about 40° C. and about 130° C.

The spray-dried particle can be fabricated with features which enhance aerosolization via dry powder inhaler devices, and lead to lower deposition in the mouth, throat and inhaler device. In addition, the spray dried particles can be fabricated with a rough surface texture to reduce particle agglomeration and improve flowability of the powder, as described below.

Particle and Nanoparticle Characteristics

The particles of the present invention are aerodynamically light, having a preferred size, e.g., a volume median geometric diameter (VMGD or geometric diameter) of at least about 5 microns. In one embodiment, the VMGD is from about 5 μm to about 15 μm. In another embodiment of the invention, the particles have a VMGD ranging from about 10 μm to about 15 μm, and as such, more successfully avoid phagocytic engulfment by alveolar macrophages and clearance from the lungs, due to size exclusion of the particles from the phagocytes' cytosolic space. Phagocytosis of particles by alveolar macrophages decreases precipitously as particle diameter increases beyond about 3 μm and less than about 1 μm (Kawaguchi et al., Biomaterials 7: 61-66, 1986; Krenis and Strauss, Proc. Soc. Exp. Med., 107: 748-750,1961; and Rudt and Muller, J. Contr. Rel., 22: 263-272,1992). In another embodiment, the particles have a VMGD of approximately 65 μm.

In addition, the nanoparticles contained within the spray dried particles have a geometric diameter of approximately less than about 1 μm, for example, from about 25 nanometers to approximately 1 μm. Such geometric diameters are small enough that the escape clearance from the body by macrophages, and can reside in the body for long periods of time. In other embodiments, the particles have a median diameter (MD), MMD, a mass median envelope diameter (MMED) or a mass median geometric diameter (MMGD) of at least 5 μm, for example from about 5 μm to about 30 μm.

Suitable particles can be fabricated or separated, for example, by filtration or centrifugation, to provide a particle sample with a preselected size distribution. For example, greater than about 30%, 50%, 70%, or 80% of the particles in a sample can have a diameter within a selected range of at least about 5 μm. The selected range within which a certain percentage of the particles must fall may be, for example, between about 5 and about 30 μm, or optimally between about 5 and about 25 μm. In one preferred embodiment, at least a portion of the particles have a diameter between about 5 μm and about 15 μm. Optionally, the particle sample also can be fabricated wherein at least about 90%, or optionally about 95% or about 99%, have a diameter within the selected range.

The aerodynamically light particles of the present invention preferably have MMAD, also referred to herein as “aerodynamic diameter,” between about 1 μm and about 10 μm. In one embodiment of the invention, the MMAD is between about 1 μm and about 5 μm. In another embodiment, the MMAD is between about 1 μm and about 3 μm. The aerodynamic diameter of such particles make them ideal for delivery to the lungs.

The diameter of the particles, for example, their VMGD, can be measured using an electrical zone sensing instrument such as a Multisizer IIe, (Coulter Electronic, Luton, Beds, England), or a laser diffraction instrument (for example, Helos, manufactured by Sympatec, Princeton, N.J.) or by SEM visualization. Other instruments for measuring particle diameter are well known in the art. The diameter of particles in a sample will range depending upon factors such as particle composition and methods of synthesis. The distribution of size of particles in a sample can be selected to permit optimal deposition within targeted sites within the respiratory tract.

Experimentally, aerodynamic diameter can be determined by employing a gravitational settling method, whereby the time for an ensemble of particles to settle a certain distance is used to infer directly the aerodynamic diameter of the particles. An indirect method for measuring the mass median aerodynamic diameter (MMAD) is the multi-stage liquid impinger (MSLI).

The aerodynamic diameter, dₐₑᵣ, can be calculated from the equation:

dₐₑᵣ=d_{g}√ρₜₐₚ

where d_{g} is the geometric diameter, for example the MMGD and ρ is the particle mass density approximated by the powder tap density.

In certain embodiments, hollow particles are formed. Two characteristic times are critical to the drying process that leads to the formation of hollow particles. The first is the time it takes for a droplet to dry and the second the time it takes for a solute/nanoparticle to diffuse from the edge of the droplet to its center. The ratio of the two describes the so-called Peclet number (Pe) a dimensionless mass transport number characterizing the relative importance of diffusion and convection (Stroock, A.D., Dertinger, S. K. W., Ajdari, A. Mezic, I., Stone, H. A. & Whitesides, G. M. Science (2002) 295, 647, 651). Thus, if the drying of the droplet is sufficiently slow (i.e., Pe<<1), solute or nanoparticles have adequate time to distribute by diffusion throughout the evaporating droplet, yielding relatively dense dried particles. On the other hand, if the drying of the droplet is very quick (i.e., Pe>>1)., then solute or nanoparticle have insufficient time to diffuse back to the center of the droplet, being collected by the drying front of the droplet. Nanoparticles tend to be trapped at the free surface of the droplet in a potential well (Pieranski, P., Phys. Rev. Lett. (1980) 45, 569-572). Capillary forces draw nanoparticles together and once in contact lock them electrostatically by Van der Waals forces (Velev, O. D., Furusawa, K. & Nagayama, K., Langmuir (1996) 12, 2374-2384, Langmuir (1996) 12, 2385-2391, Langmuir (1997) 13, 1856-1859). Nanoparticles continue to collect on the evaporating front until formation of a shell or crust in which the remaining solution is enclosed. The solvent inside the shell gasifies, and the gas escapes the shell, pushing the internal nanoparticles to the shell surface and frequently puncturing it. This last set of the drying process is referred to as the thermal expansion phase.

Particle Delivery

The particles of the present invention are pharmaceutical compositions that are administered to the respiratory tract of a patient in need of treatment, prophylaxis or diagnosis. Administration of particles to the respiratory system can be by means such as known in the art. For example, particles (agglomerates) can be delivered from an inhalation device. In a preferred embodiment, particles are administered via a dry powder inhaler (DPI). Metered-dose-inhalers (MDI), nebulizers, or instillation techniques also can be employed. Preferably, delivery is to the alveoli region of the pulmonary system, the central airways, or the upper airways.

In particular the following diseases or conditions can be treated with the pharmaceutical compositions and methods of the present invention: tuberculosis, diabetes, asthma, and acute health problems caused by chemical and biological terrorism.

Various suitable devices and methods of inhalation which can be used to administer particles to a patient's respiratory tract are known in the art. For example, suitable inhalers are described in U.S. Pat. Nos. 4,995,385, and 4,069,819 issued to Valentini et al., U.S. Pat. No. 5,997,848 issued to Patton. Other examples include, but are not limited to, the Spinhaler® (Fisons, Loughborough, U.K.), Rotahaler® (Glaxo-Wellcome, Research Triangle Technology Park, North Carolina), FlowCaps® (Hovione, Loures, Portugal), Inhalator® (Boehringer-Ingelheim, Germany), the Aerolizer® (Novartis, Switzerland), the diskhaler (Glaxo-Wellcome, RTP, NC) and others, known to those skilled in the art. Preferably, the particles are administered as a dry powder via a dry powder inhaler.

In one embodiment, the dry powder inhaler is a simple, breath actuated device. An example of a suitable inhaler which can be employed is described in U.S. patent application, entitled Inhalation Device and Method, by David A. Edwards et al., with Ser. No. 09/835,302 filed on Apr. 16, 2001. The entire contents of this application are incorporated by reference herein. This pulmonary delivery system is particularly suitable because it enables efficient dry powder delivery of small molecules, proteins and peptide bioactive agent particles deep into the lung. Particularly suitable for delivery are the unique porous particles, such as the particles described herein, which are formulated with a low mass density, relatively large geometric diameter and optimum aerodynamic characteristics. These particles can be dispersed and inhaled efficiently with a simple inhaler device. In particular, the unique properties of these particles confers the capability of being simultaneously dispersed and inhaled.

A receptacle encloses or stores particles and/or respirable pharmaceutical compositions comprising the particles. The receptacle is filled with the particles using methods as known in the art. For example, vacuum filling or tamping technologies may be used. Generally, filling the receptacle with the particles can be carried out by methods known in the art. In one embodiment of the invention, the particles that are enclosed or stored in a receptacle have a mass of at least about 5 milligrams. In another embodiment, the mass of the particles stored or enclosed in the receptacle comprises a mass of bioactive agent from at least about 1.5 mg to at least about 20 milligrams. In still another embodiment, the mass of the particles stored or enclosed in the receptacle comprises a mass of bioactive agent of at least about 100 milligrams, for example, when the particles are 100% bioactive agent.

In one embodiment, the volume of the an inhaler receptacle is at least about 0.37 cm³. In another embodiment, the volume of the inhaler receptacle is at least about 0.48 cm³. In yet another embodiment, are inhaler receptacles having a volume of at least about 0.67 cm³ or 0.95 cm³. Alternatively, the receptacles can be capsules, for example, capsules designated with a particular capsule size, such as 2, 1, 0, 00 or 000. Suitable capsules can be obtained, for example, from Shionogi (Rockville, Md.). Blisters can be obtained, for example, from Hueck Foils, (Wall, N.J.). Other receptacles and other volumes thereof suitable for use in the instant invention are also known to those skilled in the art.

Preferably, particles administered to the respiratory tract travel through the upper airways (oropharynx and larynx), the lower airways which include the trachea followed by bifurcations into the bronchi and bronchioli and through the terminal bronchioli which in turn divide into respiratory bronchioli leading then to the ultimate respiratory zone, the alveoli or the deep lung. In a preferred embodiment of the invention, most of the mass of particles deposits in the deep lung. In another embodiment of the invention, delivery is primarily to the central airways. Delivery to the upper airways can also be obtained.

In one embodiment of the invention, delivery to the pulmonary system of particles is in a single, breath-actuated step, as described in U.S. patent application Ser. Nos. 09/591,307, filed Jun. 9, 2000, and 09/878,146, filed Jun. 8, 2001, the entire teachings of which are incorporated herein by reference. In a preferred embodiment, the dispersing and inhalation occurs simultaneously in a single inhalation in a breath-actuated device. An example of a suitable inhaler which can be employed is described in U.S. patent application, entitled Inhalation Device and Method, by David A. Edwards et al., with Ser. No. 09/835,302 filed on Apr. 16, 2001. The entire contents of this application are incorporated by reference herein. In another embodiment of the invention, at least 50% of the mass of the particles stored in the inhaler receptacle is delivered to a subject's respiratory system in a single, breath-activated step. In a further embodiment, at least 5 milligrams and preferably at least 10 milligrams of a bioactive agent is delivered by administering, in a single breath, to a subject's respiratory tract particles enclosed in the receptacle. Amounts of bioactive agent as high as 15, 20, 25, 30, 35, 40 and 50 milligrams can be delivered.

Aerosol dosage, formulations and delivery systems also may be selected for a particular therapeutic application, as described, for example, in Gonda, I. “Aerosols for delivery of therapeutic and diagnostic agents to the respiratory tract,” in Critical Reviews in Therapeutic Drug Carrier Systems, 6: 273-313, 1990; and in Moren, “Aerosol dosage forms and formulations,” in: Aerosols in Medicine. Principles, Diagnosis and Therapy, Moren et al., Eds, Elsevier, Amsterdam, 1985.

Bioactive agent release rates from particles and/or nanoparticles can be described in terms of release constants. The first order release constant can be expressed using the following equations:

M₍ₜ₎=M_{(∞)}*(1−e^{-k*t}) (1)

Where k is the first order release constant. M_{(∞)} is the total mass of bioactive agent in the bioactive agent delivery system, e.g. the dry powder, and M₍ₜ₎ is the amount of bioactive agent mass released from dry powders at time t.

Equation (1) may be expressed either in amount (i.e., mass) of bioactive agent released or concentration of bioactive agent released in a specified volume of release medium.

For example, Equation (1) may be expressed as:

C₍ₜ₎=C_{(∞)}*(1−e^{-k*t}) or Release₍ₜ₎=Release_{(∞)}*(1−e^{-k*t}) (2)

Where k is the first order release constant. C_{(∞)} is the maximum theoretical concentration of bioactive agent in the release medium, and C₍ₜ₎ is the concentration of bioactive agent being released from dry powders to the release medium at time t.

Drug release rates in terms of first order release constant can be calculated using the following equations:

k=−1 n(M_{(∞)}−M₍ₜ₎)/M_{(∞)}/t (3)

Release rates of bioactive agents from particles and/or nanoparticles can be controlled or optimized by adjusting the thermal properties or physical state transitions of the particles and/or nanoparticles. The particles and/or nanoparticles of the invention can be characterized by their matrix transition temperature. As used herein, the term “matrix transition temperature” refers to the temperature at which particles are transformed from glassy or rigid phase with less molecular mobility to a more amorphous, rubbery or molten state or fluid-like phase. As used herein, “matrix transition temperature” is the temperature at which the structural integrity of a particle and/or nanoparticle is diminished in a manner which imparts faster release of bioactive agent from the particle. Above the matrix transition temperature, the particle structure changes so that mobility of the bioactive agent molecules increases resulting in faster release. In contrast, below the matrix transition temperature, the mobility of the bioactive agent particles and/or nanoparticles is limited, resulting in a slower release. The “matrix transition temperature” can relate to different phase transition temperatures, for example, melting temperature (Tₘ), crystallization temperature (T_{c}) and glass transition temperature (T_{g}) which represent changes of order and/or molecular mobility within solids.

Experimentally, matrix transition temperatures can be determined by methods known in the art, in particular by differential scanning calorimetry (DSC). Other techniques to characterize the matrix transition behavior of particles or dry powders include synchrotron X-ray diffraction and freeze fracture electron microscopy.

Matrix transition temperatures can be employed to fabricate particles and/or nanoparticles having desired bioactive agent release kinetics and to optimize particle formulations for a desired bioactive agent release rate. Particles and/or nanoparticles having a specified matrix transition temperature can be prepared and tested for bioactive agent release properties by in vitro or in vivo release assays, pharmacokinetic studies and other techniques known in the art. Once a relationship between matrix transition temperatures and bioactive agent release rates is established, desired or targeted release rates can be obtained by forming and delivering particles and/or nanoparticles which have the corresponding matrix transition temperature. Drug release rates can be modified or optimized by adjusting the matrix transition temperature of the particles and/or nanoparticles being administered.

The particles and/or nanoparticles of the invention include one or more materials which, alone or in combination, promote or impart to the particles a matrix transition temperature that yields a desired or targeted bioactive agent release rate. Properties and examples of suitable materials or combinations thereof are further described below. For example, to obtain a rapid release of a bioactive agent, materials, which, when combined, result in a low matrix transition temperatures, are preferred. As used herein, “low transition temperature” refers to particles which have a matrix transition temperature which is below or about the physiological temperature of a subject. Particles and/or nanoparticles possessing low transition temperatures tend to have limited structural integrity and be more amorphous, rubbery, in a molten state, or fluid-like.

Without wishing to be held to any particular interpretation of a mechanism of action, it is believed that, for particles and/or nanoparticles having low matrix transition temperatures, the integrity of the particle and/or nanoparticle matrix undergoes transition within a short period of time when exposed to body temperature (typically around 37 ° C.) and high humidity (approaching 100% in the lungs) and that the components of these particles tend to possess high molecular mobility allowing the bioactive agent to be quickly released and available for uptake.

Designing and fabricating particles and/or nanoparticles with a mixture of materials having high phase transition temperatures can be employed to modulate or adjust matrix transition temperatures of resulting particles and/or nanoparticles and corresponding release profiles for a given bioactive agent.

Combining appropriate amount of materials to produce particles and/or nanoparticles having a desired transition temperature can be determined experimentally, for example, by forming particles having varying proportions of the desired materials, measuring the matrix transition temperatures of the mixtures (for example by DSC), selecting the combination having the desired matrix transition temperature and, optionally, further optimizing the proportions of the materials employed.

Miscibility of the materials in one another also can be considered. Materials which are miscible in one another tend to yield an intermediate overall matrix transition temperature, all other things being equal. On the other hand, materials which are immiscible in one another tend to yield an overall matrix transition temperature that is governed either predominantly by one component or may result in biphasic release properties.

In a preferred embodiment, the particles and/or nanoparticles include one or more phospholipids. The phospholipid or combination of phospholipids is selected to impart specific bioactive agent release properties to the particles and/or nanoparticles. Phospholipids suitable for pulmonary delivery to a human subject are preferred. In one embodiment, the phospholipid is endogenous to the lung. In another embodiment, the phospholipid is non-endogenous to the lung.

The phospholipid can be present in the particles in an amount ranging from about 1 weight % to about 99 weight %. Preferably, it can be present in the particles in an amount ranging from about 10 weight % to about 80 weight %.

Examples of phospholipids include, but are not limited to, phosphatidic acids, phosphatidylcholines, phosphatidylethanolamines, phosphatidylglycerols, phosphatidylserines, phosphatidylinositols or a combination thereof. Modified phospholipids for example, phospholipids having their head group modified, e.g., alkylated or polyethylene glycol (PEG)—modified, also can be employed.

In a preferred embodiment, the matrix transition temperature of the particles is related to the phase transition temperature, as defined by the melting temperature (Tₘ), the crystallization temperature (T_{c}) and the glass transition temperature (T_{g}) of the phospholipid or combination of phospholipids employed in forming the particles. Tₘ, T_{c} and T_{g} are terms known in the art. For example, these terms are discussed in Phospholipid Handbook (Gregor Cevc, editor, 1993) Marcel-Dekker, Inc.

Phase transition temperatures for phospholipids or combinations thereof can be obtained from the literature. Sources listing phase transition temperature of phospholipids is, for instance, the Avanti Polar Lipids (Alabaster, Ala.) Catalog or the Phospholipid Handbook (Gregor Cevc, editor, 1993) Marcel-Dekker, Inc. Small variations in transition temperature values listed from one source to another may be the result of experimental conditions such as moisture content.

Experimentally, phase transition temperatures can be determined by methods known in the art, in particular by differential scanning calorimetry. Other techniques to characterize the phase behavior of phospholipids or combinations thereof include synchrotron X-ray diffraction and freeze fracture electron microscopy.

Combining the appropriate amounts of two or more phospholipids to form a combination having a desired phase transition temperature is described, for example, in the Phospholipid Handbook (Gregor Cevc, editor, 1993) Marcell-Dekker, Inc. Miscibilities of phospholipids in one another may be found in the Avanti Polar Lipids (Alabaster, Ala.) Catalog.

The amounts of phospholipids to be used to form particles and/or nanoparticles having a desired or targeted matrix transition temperature can be determined experimentally, for example by forming mixtures in various proportions of the phospholipids of interest, measuring the transition temperature for each mixture, and selecting the mixture having the targeted transition temperature. The effects of phospholipid miscibility on the matrix transition temperature of the phospholipid mixture can be determined by combining a first phospholipid with other phospholipids having varying miscibilities with the first phospholipid and measuring the transition temperature of the combinations.

Combinations of one or more phospholipids with other materials also can be employed to achieve a desired matrix transition temperature. Examples include polymers and other biomaterials, such as, for instance, lipids, sphingolipids, cholesterol, surfactants, polyaminoacids, polysaccharides, proteins, salts and others. Amounts and miscibility parameters selected to obtain a desired or targeted matrix transition temperatures can be determined as described above.

In general, phospholipids, combinations of phospholipids, as well as combinations of phospholipids with other materials, which have a phase transition temperature greater than about the physiological body temperature of a patient, are preferred in forming slow release particles. Such phospholipids or phospholipid combinations are referred to herein as having high transition temperatures. Particles and nanoparticles containing such phospholipids or phospholipid combinations are suitable for sustained action release of bioactive agents.

Examples of suitable high transition temperature phospholipids are shown in Table 2. Transition temperatures shown are obtained from the Avanti Polar Lipids (Alabaster, Ala.) Catalog.

TABLE 2

| | | Transition |
|---|---|---|
| | Phospholipids | Temperature |
| 1. | 1,2-Diheptadecanoyl-sn-glycero-3-phosphocholine | 48° C. |
| 2. | 1,2-Distearoyl-sn-glycero-3-phosphocholine (DSPC) | 55° C. |
| 3. | 1-Palmitoyl-2-stearoyl-sn-glycero-3-phosphocholine | 49° C. |
| 4. | 1,2-Dimyristoyl-sn-glycero-3-phosphate (DMPA) | 50° C. |
| 5. | 1,2-Dipalmitoyl-sn-glycero-3-phosphate (DPPA) | 67° C. |
| 6. | 1,2-Dipalmitoyl-sn-glycero-3-[phospho-L-serine] | 54° C. |
| 7. | 1,2-Distearoyl-sn-glycero-3-[phospho-L-serine] | 68° C. |
| 8. | 1,2-Distearoyl-sn-glycero-3-[phospho-rac-(1-glycerol)] | 55° C. |
| | (DSPG) | |
| 9. | 1,2-Dimyristoyl-sn-glycero-3-phosphoethanolamine | 50° C. |
| | (DMPE) | |
| 10. | 1,2-Dipalmitoyl-sn-glycero-3-phosphoethanolamine | 63° C. |
| | (DPPE) | |
| 11. | 1,2-Distearoyl-sn-glycero-3-phosphoethanolamine | 74° C. |
| | (DSPE) | |

In general, phospholipids, combinations of phospholipids, as well as combinations of phospholipids with other materials, which yield a matrix transition temperature no greater than about the physiological body temperature of a patient, are preferred in fabricating particles which have fast bioactive agent release properties. Such phospholipids or phospholipid combinations are referred to herein as having low transition temperatures. Thus, particles comprising such phospholipids can dissolve rapidly to deliver the nanoparticles contained in the particles to the target site, for example the respiratory tract or the deep lung. Examples of suitable low transition temperature phospholipids are listed in Table 3. Transition temperatures shown are obtained from the Avanti Polar Lipids (Alabaster, Ala.) Catalog.

TABLE 3

| | | Transition |
|---|---|---|
| | Phospholipids | Temperature |
| 1 | 1,2-Dilauroyl-sn-glycero-3-phosphocholine (DLPC) | −1° C. |
| 2 | 1,2-Ditridecanoyl-sn-glycero-3-phosphocholine | 14° C. |
| 3 | 1,2-Dimyristoyl-sn-glycero-3-phosphocholine (DMPC) | 23° C. |
| 4 | 1,2-Dipentadecanoyl-sn-glycero-3-phosphocholine | 33° C. |
| 5 | 1,2-Dipalmitoyl-sn-glycero-3-phosphocholine (DPPC) | 41° C. |
| 6 | 1-Myristoyl-2-palmitoyl-sn-glycero-3-phosphocholine | 35° C. |
| 7 | 1-Myristoyl-2-stearoyl-sn-glycero-3-phosphocholine | 40° C. |
| 8 | 1-Palmitoyl-2-myristoyl-sn-glycero-3-phosphocholine | 27° C. |
| 9 | 1-Stearoyl-2-myristoyl-sn-glycero-3-phosphocholine | 30° C. |
| 10 | 1,2-Dilauroyl-sn-glycero-3-phosphate (DLPA) | 31° C. |
| 11 | 1,2-Dimyristoyl-sn-glycero-3-[phospho-L-serine] | 35° C. |
| 12 | 1,2-Dimyristoyl-sn-glycero-3-[phospho-rac- | 23° C. |
| | (1-glycerol)] (DMPG) | |
| 13 | 1,2-Dipalmitoyl-sn-glycero-3-[phospho-rac- | 41° C. |
| | (1-glycerol)] (DPPG) | |
| 14 | 1,2-Dilauroyl-sn-glycero-3-phosphoethanolamine | 29° C. |
| | (DLPE) | |

Phospholipids having a head group selected from those found endogenously in the lung, e.g., phosphatidylcholine, phosphatidylethanolamines, phosphatidylglycerols, phosphatidylserines, phosphatidylinositols or a combination thereof are preferred.

The above materials can be used alone or in combinations. Other phospholipids which have a phase transition temperature no greater than a patient's body temperature, also can be employed, either alone or in combination with other phospholipids or materials.

As used herein, the term “nominal dose” means the total mass of bioactive agent which is present in the mass of particles targeted for administration and represents the maximum amount of bioactive agent available for administration. In addition, the terms “a,” “an,” and “the” include plural referents unless the content clearly dictates otherwise.

Guidance for making the particles of the present invention can also be found in U.S. Provisional Patent Applications entitled “Particulate Compositions For Improving Solubility of Poorly Soluble Agents” (application Ser. No. 60/331,810 filed Nov. 20, 2001); and “High Surface Area Particles for Inhalation” (application Ser. No. 60/331,708 filed Nov. 20, 2001), the entire contents of which are hereby incorporated by reference. Additional guidance can be found in U.S. patent applications entitled “Particulate Compositions For Improving Solubility of Poorly Soluble Agents” (Atty. Docket Number 2685-2014-001, filed Nov. 20, 2002); and “Improved Particulate Compositions for Pulmonary Delivery” (Atty. Docket Number 2685-2009-001, filed Nov. 20, 2002), the entire contents of which are hereby incorporated by reference.

The present invention will be further understood by reference to the following non-limiting examples.

### EXEMPLIFICATION

### EXAMPLE 1

Materials

1,2-Dipalmitoyl-sn-glycero-3-phosphocholine (DPPC, molecular weight (MW)=734.05) was purchased from Avanti Polar Lipids, Inc. (Alabaster, Ala.) and 1,2-Dimyristoyl-sn-glycero-3-phosphoethanolamine (DMPE, MW=635.86) was purchased from Genzyme (Cambridge, Mass.), both with a purity approximately 99%. Lactose Monohydrate (4-O-beta-Galactopyranosyl-D-glucose, MW=360.31) and ammonium bicarbonate were purchased from Spectrum laboratory products (New Brunswick, N.J.) with a purity of approximately 99%. Bovine Serum Albumin fraction V (MW=66000, BSA approximately 99%), Insulin (MW approximately 6000), Poly(vinyl alcohol) (PVA, MW=13000-23000, 87-89% hydrolyzed, purity of approximately 99%), Trizma base and dichloromethane (purity of approximately 99.9%) were purchased from Sigma-Aldrich (St Louis, Mo.). Distilled water USP grade was purchased from B. Braun Medical Inc. (Irvine, Calif.) and ethanol USP grade was obtained from PharmCo (Brookfield, Conn.). Carboxylate modified white polystyrene latex beads (CML) were purchased from Interfacial Dynamics Corporation (IDC, Portland, Oreg.) with diameters of 25±3,170±8 and 1000±66 nm. These beads were provided in solution in water with respective weight concentrations of approximately 3.1%, 4.5% and 4.2%. Nyacol 9950 colloidal silica (diameter approximately 100 nm) was purchased from EKA Chemicals (Marietta, Ga.) with a weight concentration of 50% in water. Polystyrene broad distribution (MW=6800, polydispersity index=1.17) was purchased from Polymer Source (Dorval, Quebec, Canada). Estradiol micronized powder was purchased from Spectrum laboratory products (New Brunswick, N.J.) with a purity of approximately 99%.

### EXAMPLE 2

Preparation of Solutions For Spray-Drying

DPPC-DMPE-Lactose (With or Without Beads)

0.6 g of DPPC was dissolved in 700 ml ethanol upon magnetic stirring. Then 0.2 g DMPE was added to this solution. In order to dissolve the DMPE, the solution was placed in a thermostated bath at 60° C. with magnetic stirring until it was clear. 0.210 g lactose monohydrate was dissolved in 300 ml water upon magnetic stirring. Both solutions were then mixed together (using a magnetic stirrer). The resulting mixture was then ready for spray-drying. At this point the desired amount of beads (CML polystyrene latex) was added directly in the mixture. In the case of the silica colloidal beads, water was replaced by 25 mM Tris buffer (pH=9.25) to ensure colloidal silica stability. The buffer was prepared by solubilizing 2.93 g of Trizma base in a liter of water, the pH was then adjusted to 9.25 by adding HCl 1N. The buffer containing lactose was mixed with the lipids/ethanol solution as described above, and then desired amount of colloidal silica was added. In the case of laboratory-designed PS beads, 0.210 g lactose monohydrate was added to 300 ml of water already containing the beads (see below for laboratory-designed PS beads preparation), and then mixed with the lipids/ethanol solution.

BSA (With or Without Beads)

3.255 g BSA and 0.245 g sodium phosphate monobasic were dissolved in 800 ml water upon magnetic stirring. The solution pH was adjusted to 7.4 by adding KOH (1N). 15 g ammonium bicarbonate was then dissolved in this solution. 200 ml ethanol was mixed with the resulting solution until homogenization. At this point the desired amount of beads (CML polystyrene latex) was added directly into the solution.

Insulin (With or Without Beads)

The pH of 400 ml of water was first adjusted to 2.5 with HCl (1N). Then, 1.0 g insulin was dissolved in the water. The pH was then adjusted to 7 with NaOH (1N) until the solution became clear. At this point, the desired amount of beads (CML polystyrene latex) was added directly into the solution. 600 ml of ethanol was also prepared and set aside for spray-drying.

### EXAMPLE 3

Preparation of Polystyrene Beads

Laboratory-designed polystyrene (PS) beads were prepared with an oil-in-water solvent evaporation technique based on a patent of Vanderhoff et al. (U.S. Pat. No. 4,177,177, the entire teachings of which are hereby incorporated by reference). Briefly, 2.8 g PVA was dissolved in 420 ml water (using a magnetic stirrer and heat). 0.5 g PS was then dissolved in 50 ml dichloromethane. To encapsulate estradiol in the beads, 0.03 g estradiol was dissolved in 1.0 ml methanol and then mixed with the dichloromethane/PS solution. Alternatively, 0.03 g estradiol can be directly dissolved in the dichloromethane/PS solution. The organic solution was then emulsified in the aqueous phase with a homogenizer IKA at 20000 RPM for 10 minutes. The organic solvent was then removed by evaporation by leaving the emulsion to stir (using a magnetic stirrer) overnight with slight heating (40-60° C.). Alternatively, the organic solvent can be removed without heating, i.e., at room temperature.

### EXAMPLE 4

Spray-Drying Conditions

All solutions were spray-dried on a NIRO Atomizer Portable spray drier (Columbus, Md.). Compressed air with variable pressure (1 to 5 bars) ran a rotary atomizer located above the dryer. Spray-dried particles are collected with a 6 inch cyclone. Others conditions depend on formulations, as described in further detail below.

DPPC-DMPE-Lactose

Two different spray drying conditions were used to generate DPPC-DMPE-lactose particles. The first spray drying conditions (SD1) were the following: the inlet temperature was fixed at 95° C.; the outlet temperature was approximately 53° C.; a V24 wheel rotating at 33000 RPM was used; the feed rate of the solution was 40 ml/min; and the drying air flow rate was 98 kg/h. The second spray drying conditions (SD2) were the following: the inlet temperature was fixed at 110° C.; the outlet temperature was approximately 46° C.; a V24 wheel rotating at 20000 RPM was used; the feed rate of the solution was 70 ml/min; and the drying air flow rate was 98 kg/h.

BSA

The spray-drying conditions for generating spray dried particles containing BSA were the following: the inlet temperature was fixed at 118° C.; the outlet temperature was approximately 64° C., a V4 wheel rotating at 50000 RPM was used; the feed rate of the solution was 30 ml/min and the drying air flow rate was 100 kg/h.

Insulin

The spray-drying conditions for making spray dried particles containing insulin were the following: the inlet temperature was fixed at 135° C.; the outlet temperature was around 64° C.; a V4 wheel rotating at 50000 RPM was used; the feed rate of the aqueous solution was 40 ml/min, whereas the feed rate of the ethanol was 25 ml/min (the two solutions were statically mixed just before being sprayed); and the drying air flow rate was 98 kg/h.

### EXAMPLE 5

Characterization of the Spray-Dried Particles

The geometric diameter of the spray-dried particles was measured by light scattering using a RODOS (Sympatec, Lawrenceville, N.J.), with an applied pressure of 2 bars.

As described above, the mass mean aerodynamic diameter (MMAD) (dₐₑᵣ) is related to the actual sphere diameter d_{g} by the formula:

dₐₑᵣ=d_{g}√ρₜₐₚ

where ρ is the particle density (U.S. Pat. No. 4,177,177). The mass mean aerodynamic diameter (MMAD) was measured with an Aerosizer™ (TSI, St Paul, Minn.), this apparatus is based on a time of flight measurement. Scanning electromicroscopy (SEM) was performed as follows: Liquid samples were deposited on double side tape and allowed to dry in an oven at 70° C. Powder samples were sprinkled on the tape and dusted. In the two cases, samples were coated with a gold layer using a Polaron SC7620 sputter coater (90 s at 18 mA).

Scanning Electron Microscopy (SEM) was performed either on a PSEM (Aspex Instruments, Dellmont, Pa.) 20 kV with a filament current of 15 mA or on a LEO 982 operating between 1 kV and 5 kV with a filament current of approximately 0.5 mA. Light scattering experiments were performed on a ALV DLS/SLS-5000 spectrometer/goniometer (ALV-Laser GmbH, Langen, Germany). This set-up consists of an argon-ion laser, beam steering optics, attenuator, sample vat, detection optics and photodiodes to measure incident intensity. The sample was placed in a quartz vat filled with toluene. The temperature of the vat was regulated by a thermostated bath with an accuracy of ±0.1K. Temperature was fixed at 298K.

The intensity autocorrelation function was measured at different angles between 30 and 120 degrees. Each angle θ corresponds to a different wave vector q:q=4 nπ sin(θ)/λ, where n is the index of the solvent and λ is the wavelength of light. Assuming that the intensity autocorrelation function is a single exponential decay with characteristic time τ, τ is related to the diffusion coefficient D of the beads by: t⁻¹=Dq2. The slope of the variation of t⁻¹ versus q² fitted by a straight line is D. The hydrodynamic radius R of the beads could then be deduced from the diffusion coefficient D using the Stokes-Einstein formula:

D₀=k_{B}T/6πηR

where k_{B} is the Boltzman constant and η the viscosity of the solvent. Laboratory-designed PS beads were diluted in water to eliminate multiple scattering. UV-Spectrophotometry was performed on a Perkin-Elmer spectrophotometer. Solutions were put in 1 cm optical path quartz Hellma cells (Müllheim, Germany).

### EXAMPLE 6

Preparation of DPPC-DMPE-Lactose Particles Containing Different Concentrations of CML Polystyrene Beads

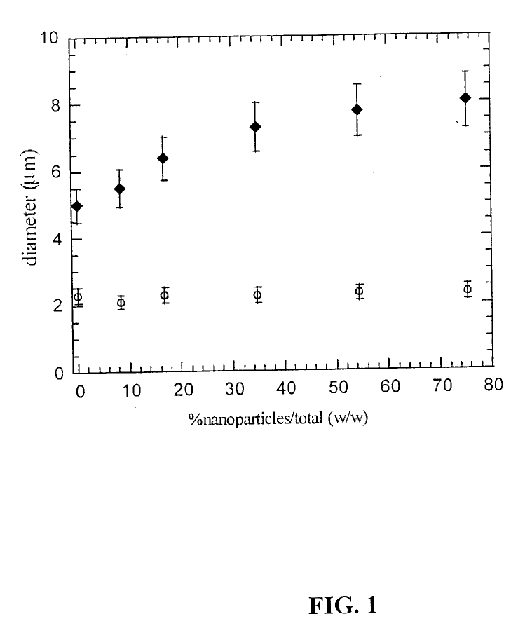

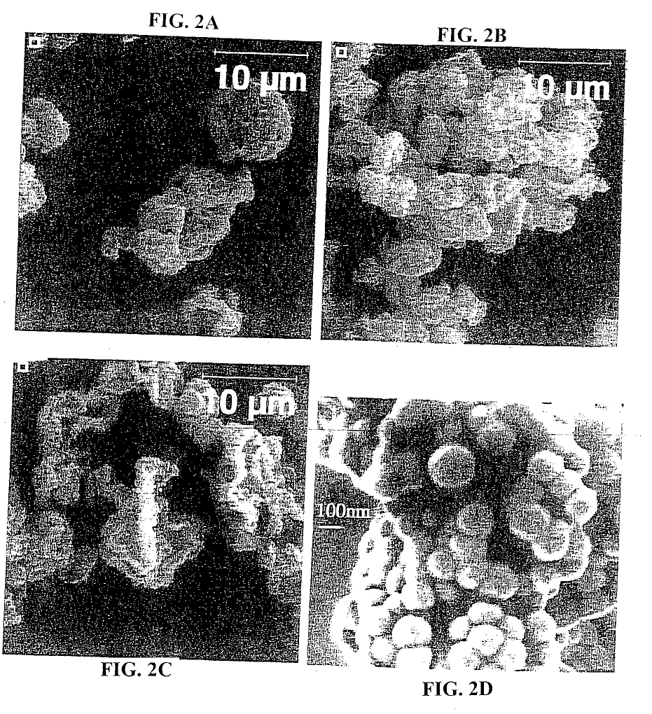

A solution of DPPC-DMPE-lactose with different concentrations of 170 nm CML polystyrene beads, as described above, was spray dried according conditions SD1. The concentration of beads spray dried into the particles ranges from 0% to approximately 75%. The geometric diameter increased with increasing concentration of beads in the particles. In contrast, the MMAD remained steady (FIG. 1). SEM pictures presented in FIGS. 2A-2D (which shows spray dried particles with and without beads) indicated that beads were incorporated in the porous particles. Importantly, adding beads to the spray-dried particles lead to larger, lighter, and therefore more flowable and aerosizable powders. In addition, as shown in FIGS. 2B-2D, the porosity of the bead-containing particles is apparent.

### EXAMPLE 7

Preparation of Spray-Dried Particles Containing Different Nanoparticle Sizes

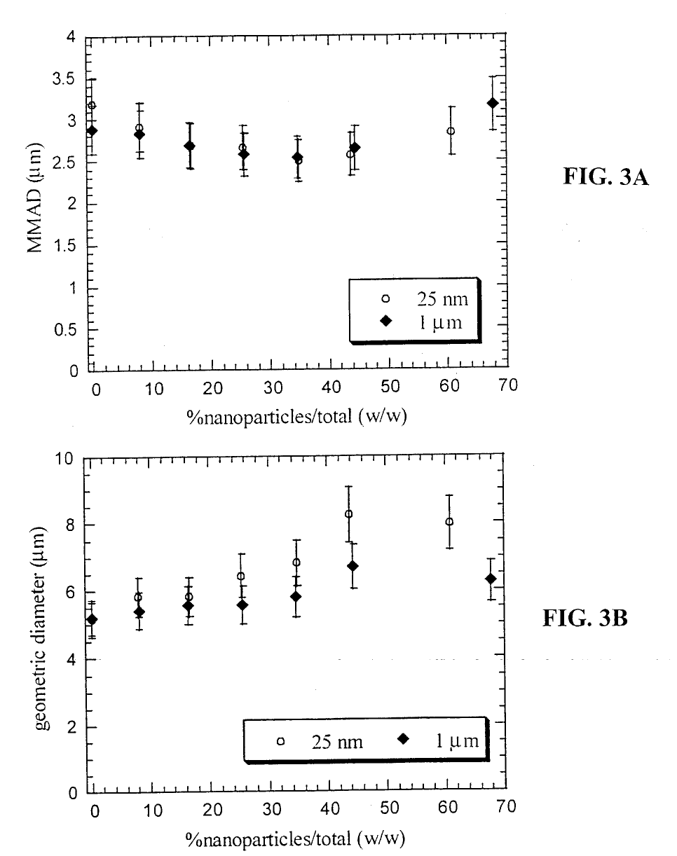

Spray-dried particles containing beads of different sizes were also generated. In particular, particles containing 25 nm CML beads and 1 micron CML beads were spray dried according to conditions SD1 described above. Relatively large, porous spray-dried particles containing each of the bead sizes were successfully produced. Regardless of bead size, the mass mean aerodynamic diameter remained fairly stable, between 2 and 3.5 microns (FIG. 3A). In contrast, in the case of particles produced to contain 25 nm beads and 1 micron beads, an increase of the geometric diameter was observed as the concentration of beads in the particles was increased (FIG. 3B). While this trend was less striking for particles produced to contain the 1 micron beads, the trend, nevertheless was observed (FIG. 3B). Thus, ability to prepare spray dried particles containing up to 70% beads is independent of the size of the beads.

### EXAMPLE 8

Effect of Various Spray Drying Conditions on Particle Formation

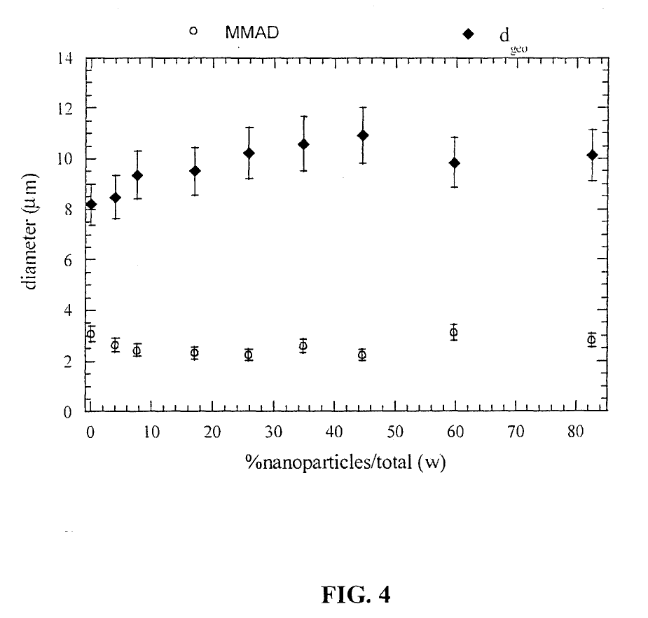

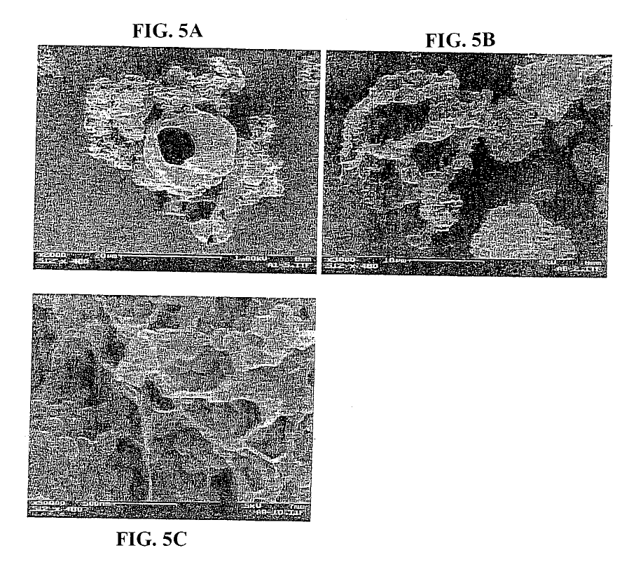

The effect of the spray drying conditions on particle geometric diameter and aerodynamic diameter was also investigated. The same solution of DPPC-DMPE-lactose in ethanol/water was spray dried according to conditions SD2, with different concentrations (up to 82%) of 170 nm diameter CML beads. As shown in FIG. 4, the same trends of an increase in geometric diameter with increasing concentration of beads and a steady aerodynamic diameter with increasing concentration of beads were observed for particles generated using SD2 conditions. SEM pictures of these particles showed that they become more crumpled, reflecting a more porous structure, as the bead concentration increased (FIGS. 5A and 5B). Closer examination of the particles indicated that beads were incorporated in them (FIG. 5C), similar to the results of particles generated using SD1 conditions.

The results of an increase in geometric diameter of spray dried particles with increasing concentration of beads incorporated into the particles, while the aerodynamic diameter remained steady regardless of concentration of beads can be explained as follows. When the sprayed droplets of solution dry, a shell of solutes forms at the droplets surface the presence of the beads may lead to an earlier formation of a more rigid shell. Thus the spray dried particles have a larger geometric diameter. However the solid content concentration of each droplet remains the same and so does the MMAD. One factor that may affect the formation of the particles is that the nanoparticles are likely to contribute to the earlier formation of the spray dried particles by being an already preformed particle.

### EXAMPLE 9

Preparation of Spray Dried Particles Using Different Nanoparticles

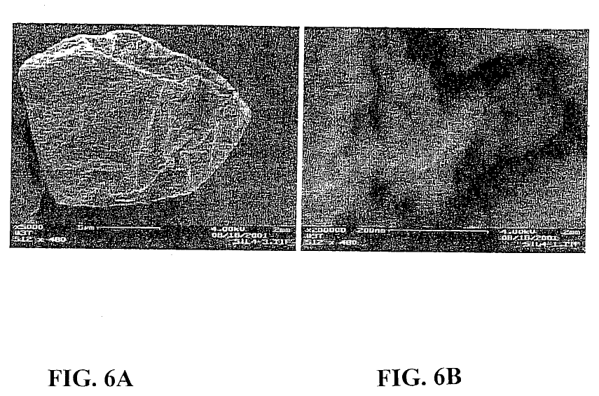

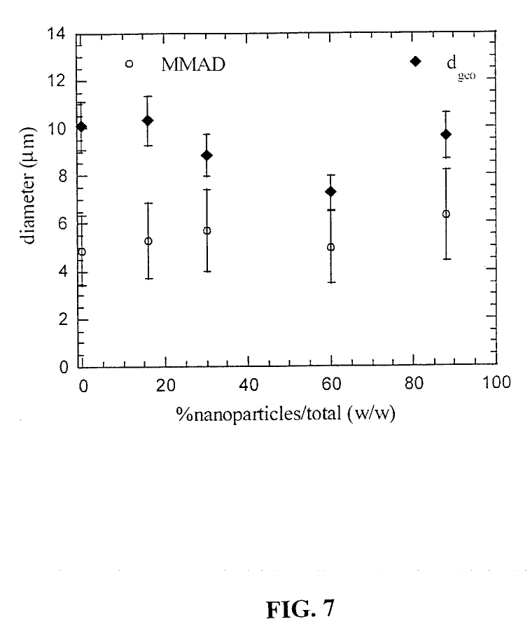

To demonstrate that the inclusion of beads in lipid spray dried particles does not depend on the surface chemistry of the beads or on the fact that polystyrene is a polymer, spray dried particles were created in which CML polystyrene beads were replaced with different beads, colloidal silica beads, which are not polymers, as described above. As in the previous experiments, the silica concentration in the spray dried particles was progressively increased. Spray dried particles containing up to 88% beads (w/w) (FIGS. 6A and 6B) were successfully prepared. However, replacing water used with the CML beads with the Tris buffer used with the colloidal beads did perturb the physical properties of the particles spray-dried without beads: particles were less porous than those made from water (aerodynamic diameter was approximately 5 microns and the geometric diameter was approximately 10 microns). Therefore the effect on the MMAD and geometric diameter of spray dried particles containing silica concentration is quite different from the effect of on the MMAD and geometric diameter of spray dried particles containing CML beads. Both the MMAD and the geometric diameter are almost constant (FIG. 7).

### EXAMPLE 10

Effect of Additive on Particle Formation

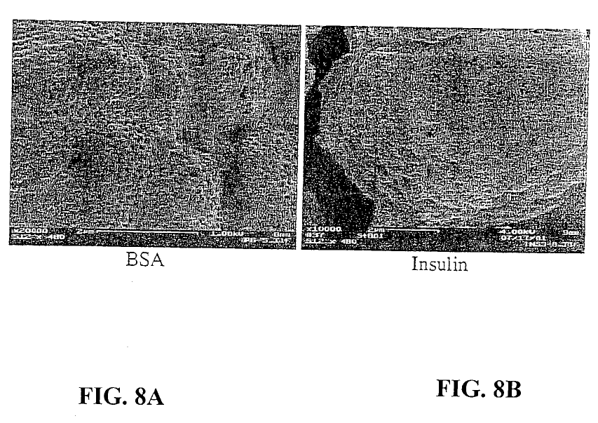

The dependence of lipidic particles for the inclusion of beads into spray dried particles was also investigated. To confirm that the inclusion of beads in spray dried particles was not dependent on the inclusion of lipidic particles, solutions of BSA and insulin, as described above, were spray dried with different concentrations of CML polystyrene beads (diameter 170 nm). Similarly to the particles containing lipids, particles containing other additives can contain up to 80% beads (w/w) as demonstrated by SEM images (FIGS. 8A and 8B). These experiments demonstrate that the ability to spray dry particles containing up to 80% beads is independent of the initial components or additives (e.g., lipids, proteins, sugars, polymers).

### EXAMPLE 11

Dissolution of Particles and Release of Nanoparticles

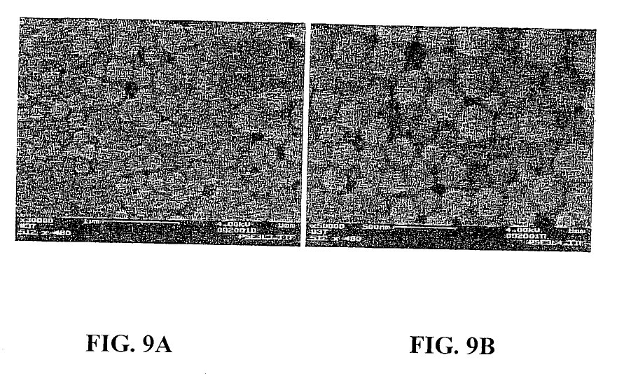

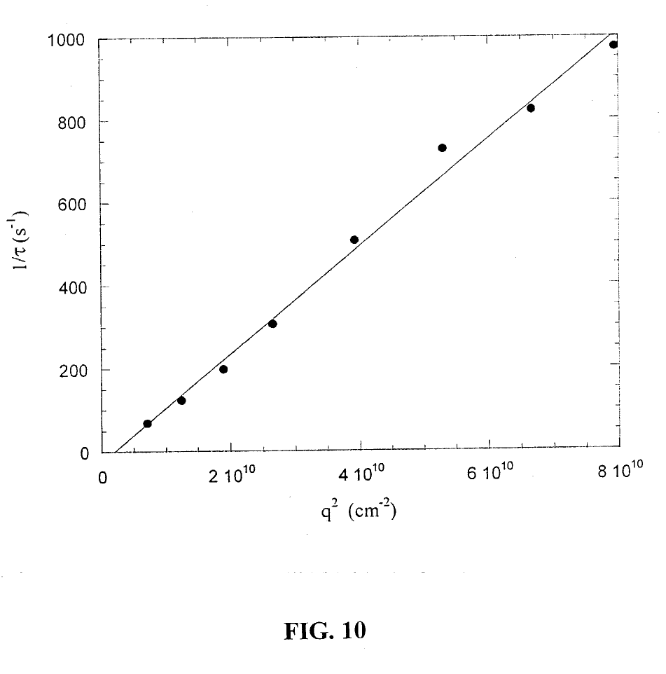

The laboratory-designed polystyrene beads prepared as described above were characterized by light scattering and SEM. The SEM images show polydisperse spheres whose diameter can be estimated between 125 and 500 nm (FIGS. 9A and 9B). Light scattering measurements give a diffusion coefficient of 1.3±0.1 cm2.s⁻¹ when data are fitted by a single exponential decay in first approximation (FIG. 10). This diffusion coefficient corresponds to a hydrodynamic diameter of approximately 370±30 nm, which is in good agreement with the SEM pictures.

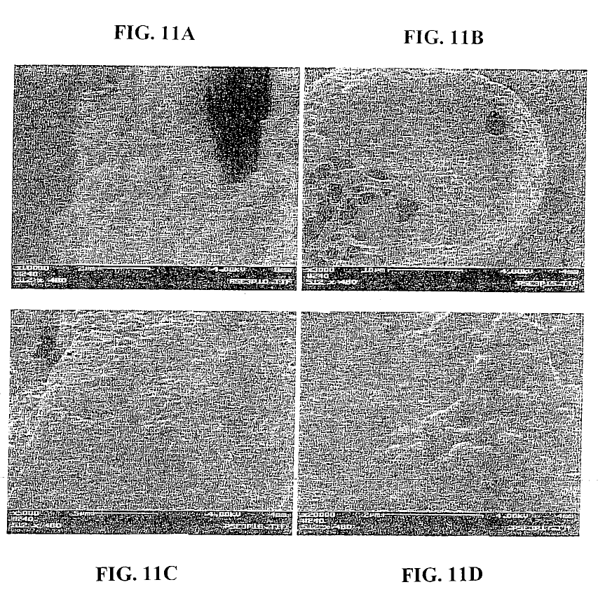

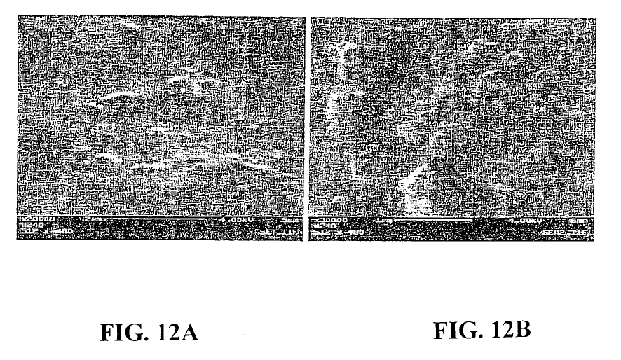

A DPPC-DMPE-lactose solution containing laboratory-designed beads was spray-dried according to conditions SD2. SEM pictures allowed for the distinction of the beads in the spray dried particles to be made (FIG. 11). Redissolution of the powder was performed in a mixture of 70/30 ethanol/water (v/v) and in pure ethanol. This solution was dried to perform SEM. Even when the powder precipitated (e.g., using 70/30 ethanol/water), SEM pictures showed distinctly sub micron size spheres very similar to the beads before spray drying (FIG. 12). Such experiments indicate that dissolution of the spray-dried particles in the lungs will release the nanoparticles. Because the bead size is very small, the beads can escape clearance from the body and therefore deliver bioactive agents for longer periods of time, or more effectively.

### EXAMPLE 12

Release of Estradiol From Nanoparticles

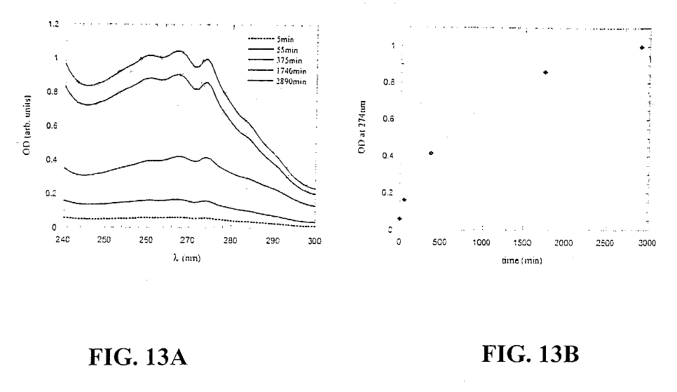

Release of the estradiol from the laboratory-designed beads was measured using spectrophotometry as follows. The solubility of 3.5 mg estradiol in 40 ml ethanol was first examined; after sonication (30 s) and stirring (several minutes) the solution was clear, indicating that estradiol is soluble in ethanol. Next, 1 ml of the beads solution (0.2 mg estradiol, 3.2 mg PS and 15.5 mg PVA) was dried at 60° C. overnight. Ethanol was then added (10 ml) onto the dry beads and the solution was put under magnetic stirring. The UV-spectrum (240-300 nm) of this solution was taken at different times, as indicated in FIG. 13A. Spectrophotometric analysis showed three peaks whose intensity increased with time. The measured optical density of the 274 nm peak was plotted versus time in FIG. 13B. As shown in FIG. 13B, the OD still increased with time over a period of 2 days. This indicated a sustained release of estradiol from the beads.

### EXAMPLE13

In Vivo Release of Estradiol From Nanoparticles

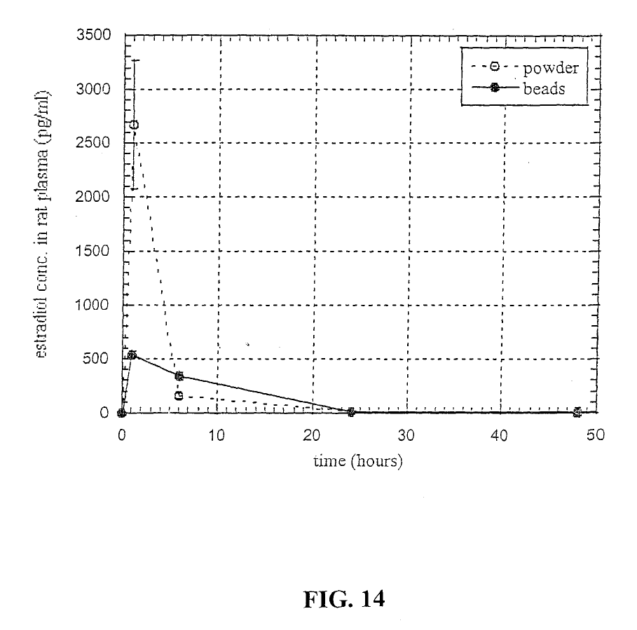

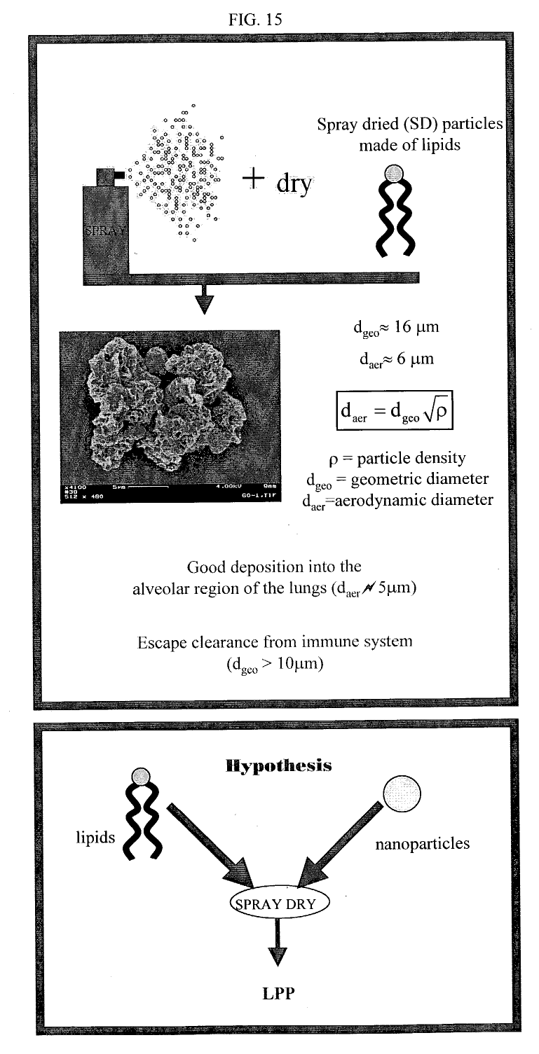

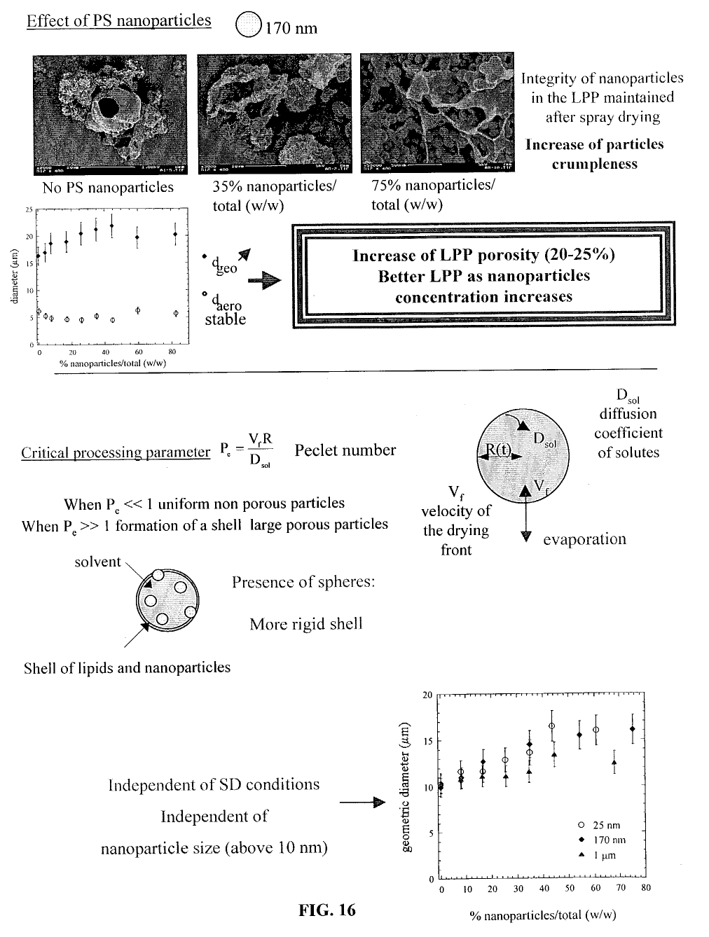

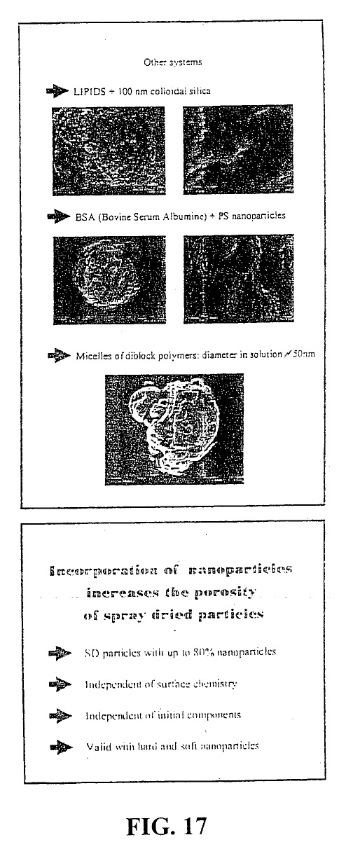

To test in vivo whether the laboratory designed PS beads slowly released estradiol, rats were administered one of two estradiol formulation by subcutaneous injection. The two formulations were: a DPPC-DMPE-lactose powder containing 1.08% estradiol resuspended in 1 ml of saline solution as a control, and a liquid solution of estradiol-loaded PS nanoparticles (concentration of estradiol=0.2029 mg/ml) (0.1 ml was added to 0.9 ml of saline solution). The nominal dose of estradiol injected to each rat was approximately 10 mg. Injections were performed on 4 rats per formulation. Plasma estradiol concentrations were measured at different times (between 0 and 48 hours). As shown in FIG. 14, a rapid elevation of the estradiol concentration in both cases just after injection was observed. Of note, the burst of estradiol is lower for the beads compared to the powder. The estradiol concentration in rats administered powder then decreased sharply over time. In contrast, estradiol was released from the beads in a more sustained manner over a longer period of time. Thus, particles containing bioactive agent-loaded PS beads will lead to a more sustained release than direct administration of the bioactive agent.

### EXAMPLE 14

Preparation of Large Porous Nanoparticles (LPNP) Containing Hydroxypropylcellulose

Materials and Methods

(Nanoparticles=(NP); Large Porous Particles=(LPP); Large Porous Nanoparticles Aggregates=(LPNP))

Materials

Hydroxypropylcellulose (MW approx. 95000), sodium phosphate monobasic monohydrate (MW=137.99) was purchased from Spectrum laboratory products (New Brunswick, N.J.) with a purity >99%.

Preparation of the Solutions for Spray-Drying:

Pure nanoparticles solution: A mixture of ethanol and water (70/30 v/v) was prepared: where the desired volume of nanoparticles (suspended in water) was added.

Lactose solution: 1 g of lactose was dissolved in 300 ml water, then 700 ml ethanol were added. Nanoparticles were then added directly to the resulting solution.

Hydroxypropylcellulose solution: 1 g of hydroxypropylcellulose was dissolved in 300 ml water, then 700 ml ethanol were added. Nanoparticles were then added directly to the resulting solution.

Spray-Drying Conditions:

Conditions termed SD2, as described herein, were used for all the solutions described above (Tinlet=110° C., Toutlet around 45° C., 20000 RPM, 70 ml/min).

Characterization of the Spray-Dried Powders:

Fine Particle Fraction (n=3) was used to characterize the SD particles containing only 170 nm nanoparticles.

Results

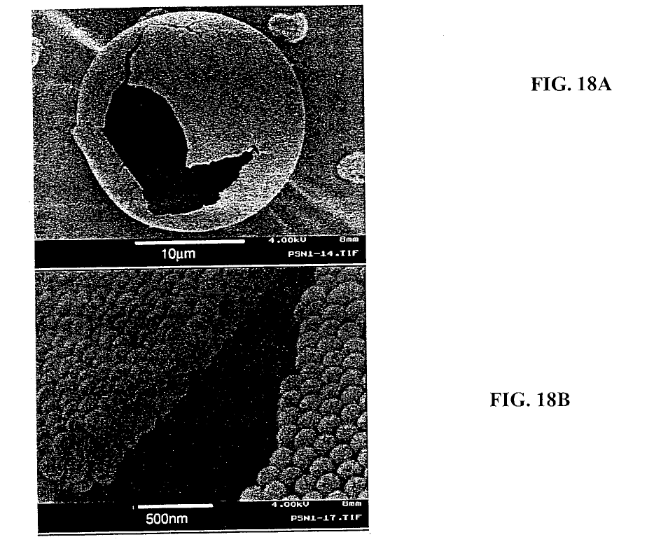

A solution of ethanol/water (70/30 in volume) was spray dried according to conditions SD2 containing carboxylate modified latex (“CML”) polystyrene beads (170 nm, 2.3 mg/ml). The SEM pictures show that the powder is composed of rather large particles compared to the initial nanoparticles. Their size in the range between 5 and 25 μm. Some of the particles (approximately 5-10%) present a rather interesting feature: a part of them is broken showing that the particle is hollow. A typical hollow particle is presented in FIGS. 18A and 18B. A zoom on the particle surface indicates that this particle is a hollow sphere whose shell is composed of the nanoparticles. The geometric diameter d_{geo} is 21 μm whereas the thickness of the shell t is about 400 nm (˜3 layers of nanoparticles). From this measurement, the aerodynamic diameter can be calculated by estimating the normalized density the following way: the geometric volume is πd³_{geo}/6, the volume occupied by the shell is π[d³_{geo}−(d_{geo}−2t)³]/6, the normalized density ρ is thus the ratio of the volume of the shell by the volume of the sphere. From the pictures presented in FIG. 18, we get ρ=0.11 and dₐₑᵣ=7 μm. The measured geometric diameter is d=6±2 μm. The results given by fine particle fraction measurement are the following: 24% of the particles have an aerodynamic diameter smaller than 5.6 μm and 15% have an aerodynamic diameter smaller than 3.4 μm.

Two characteristic times are critical to the drying process that leads to the formation of these hollow particles. The first is the time it takes for a droplet to dry and the second the time it takes for a solute/nanoparticle to diffuse from the edge of the droplet to its center. The ratio of the two describes the so-called Peclet number (Pe) a dimensionless mass transport number characterizing the relative importance of diffusion and convection (Stroock, A. D., Dertinger, S. K. W., Ajdari, A. Mezic, I., Stone, H. A. & Whitesides, G. M. Science (2002) 295, 647, 651). Thus, if the drying of the droplet is sufficiently slow (i.e., Pe<<1), solute or nanoparticles have adequate time to distribute by diffusion throughout the evaporating droplet, yielding relatively dense dried particles. On the other hand, if the drying of the droplet is very quick (i.e., Pe>>1)., then solute or nanoparticle have insufficient time to diffuse back to the center of the droplet, being collected by the drying front of the droplet. Nanoparticles tend to be trapped at the free surface of the droplet in a potential well (Pieranski, P., Phys. Rev. Lett. (1980) 45, 569-572). Capillary forces draw nanoparticles together and once in contact lock them electrostatically by Van der Waals forces (Velev, O. D., Furusawa, K. & Nagayama, K., Langmuir (1996) 12, 2374-2384, Langmuir (1996) 12, 2385-2391, Langmuir (1997) 13, 1856-1859). Nanoparticles continue to collect on the evaporating front until formation of a shell or crust in which the remaining solution is enclosed. The solvent inside the shell gasifies, and the gas escapes the shell, pushing the internal nanoparticles to the shell surface and frequently puncturing it. This last set of the drying process is referred to as the thermal expansion phase.

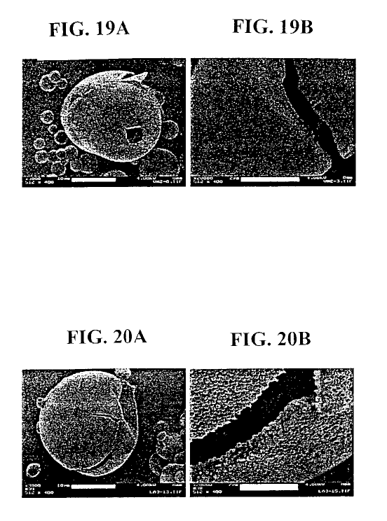

The process of LPNP creation works equally for smaller NP sizes as illustrated by our creation of LPNPs using the conditions SD2 with 25 nm nanoparticles (2.3 g/l). The SEM photos of FIGS. 19A and 19B show similar LPNP particles structure as obtained with 170 nm nanoparticles: a coexistence of large broken hollow shells and smaller rather dense particles. Shell thickness in 25 nm NP case is approximately 200 nm (i.e. 8 layers) and the geometric diameter is around 20 μm, leading to a normalized density of 0.056: the calculated aerodynamic diameter is then around 5 μm. These pictures also clearly prove that some gas is escaping from the inside by breaking the shell. Spray-drying larger nanoparticles (i.e., as large as 1 μm) does not, however, produce LPNP, as the wall formation is naturally hindered in the limit as the size of the suspended particles tend toward the size of the dried particles.

The role of the Peclet number in the formation of the LPNPs is aptly illustrated by introducing a second non-volatile species, such as lactose, a commonly spray-dried material. Lactose (1 g/l in 70/30 ethanol/water (v/v)) spray-dries (using conditions SD2) into relatively dense, non porous particles of aerodynamic diameter is 3±1 μm and geometric diameter of 4±0.5 μm (note the near coincidence of geometric and aerodynamic diameters, implying a particles mass density near unity). Adding 70% by weight polystyrene nanoparticles (170 nm) to the lactose in solution produces LPNPs, finally flowing with aerodynamic diameter 4 μm±2 μm and geometric diameter d=8±3 μm (FIGS. 20A and 20B).

The Peclet number of lactose and nanoparticles can be compared as follows: Assuming a spherical evaporating droplet of initial radius R, the Peclet number can be expressed as, Pe=R²_{/}(t_{d}Dₛₒₗ), where t_{d} is the drying time of the droplet and Dₛₒₗ the diffusion coefficient of the solute or nanoparticle species of interest. Dₛₒₗ can be estimated from the stokes-Einstein equation, Dₛₒₗ=k_{B}T/(6πηR_{H}), where k_{B} is the Boltzman constant, η the viscosity of the solvent, T the temperature and R_{H} the hydrodynamic radius of the solute or nanoparticle. Noting characteristic time (t_{d}=1 s) and droplet radius (R=45 μm) and that the hydrodynamic diameter of a lactose molecule is around 1 nm, one obtains Pe˜10 (lactose) and Pe˜2000 (PS nanoparticles) for a mixture of ethanol/water 70/30 (possessing a viscosity of 2.3 cP). Thus, in the case of the NPs, diffusive motion of nanoparticles is far slower than convective motion in the drying droplet, producing a thin walled LPNP structure, whereas in the case of the lactose (Pe˜10) convection and diffusion times are similar and hence spray-dried particles are relatively dense.

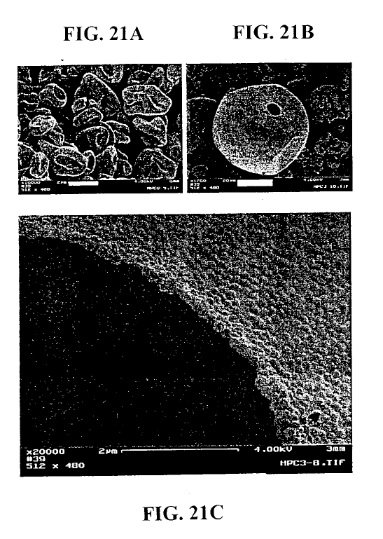

LPNPs were formed with other molecular species too. In place of the lactose, LPNPs were formed with polystyrene NPs using hydroxypropylcellulose (see FIGS. 21A, 21B, and 21C). Without nanoparticles the spray-dried particles are small and aggregate together. Because of aggregation the aerodynamic and geometric diameter measurement are not reliable but the size can be obtained from SEM pictures (around 1-2 μm). The addition of polystyrene nanoparticles to the solution before spray-drying allows to observe the coexistence of small dense particles and large hollow spheres with larger diameter and thinner shell than with lactose (for example: d=53 μm, t≈350 nm, thus ρ=0.045 and the aerodynamic diameter is 11 μm). The large particles also seem less brittle with hydroxypropylcellulose than with lactose.

### EXAMPLE 15

Formation of Nanoparticles During the Spray Drying Process

It has been observed that formation of nanoparticles can take place during the spray-drying process. Rifampicin was solubilized in 10 to 20 ml of chloroform and this solution was added to an ethanol solution containing the lipids DPPC and DMPE (700 ml) as indicated in Table 4. The resulting solution was mixed with a water solution (300 ml) containing lactose just before spray drying. The compositions of the solutions are presented in Table 4.

TABLE 4

| | % w/w | | |
|---|---|---|---|
| | A | B | C |
| DPPC | 48 | 36 | 24 |
| DMPE | 16 | 12 | 8 |
| lactose | 16 | 12 | 8 |
| RIFAMPICIN | 20 | 40 | 60 |
| Yield in % | 30% | 33% | 44% |

Solutions were spray dried according to the following conditions: the inlet temperature was 115° C. and the outlet temperature approximately 52° C. The atomizer spin rate was 20000 RPM, using a V24 wheel. The liquid feed rate was 65 ml/min and the drying gas flow rate was around 98 kg/hr.

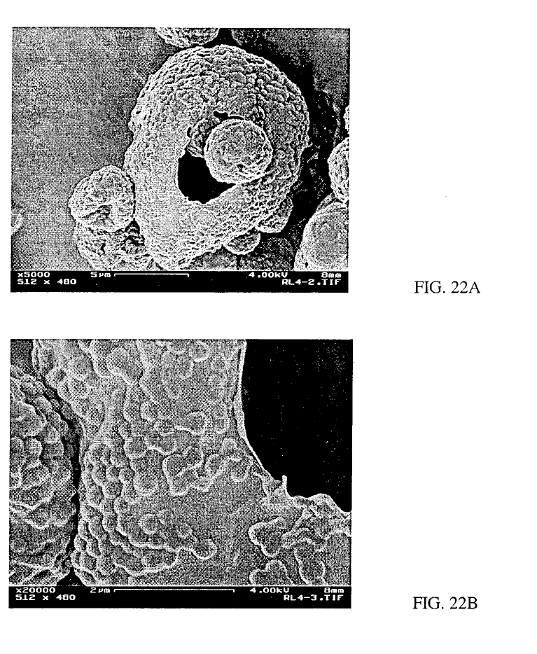

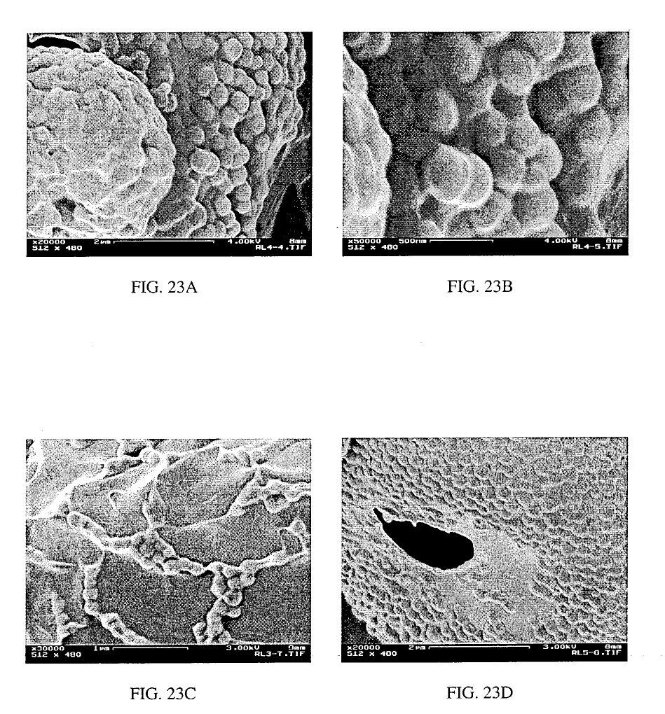

The resulting powders were examined using SEM FIGS. 22A-22B, and 23A-23D. Some nanoparticles formed spontaneously either before spray-drying or during the spray-drying process. These nanoparticles were observable in formulations A, B and C, when Rifampicin and lipids coexisted in the formulation. They appeared relatively monodisperse with a mean size between 300 and 350 nm. The concentration of nanoparticles increased with rifampicin concentration.

In order to investigate the origin of the nanoparticles observed, the following solutions were spray-dried:

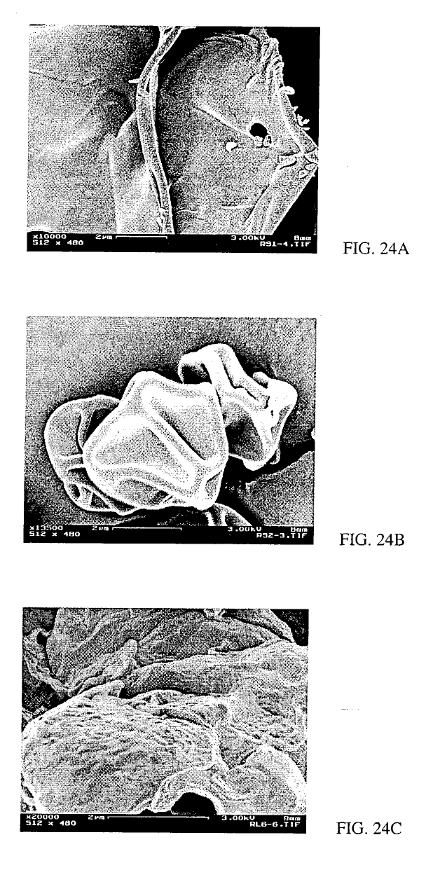

1) A solution of Rifampicin alone in a mixture of ethanol/water (70/30 v/v) (with 1% chloroform), using the same spray drying conditions as described earlier in this Example. Formation of nanoparticles was not observed (FIG. 24A).

2) A solution of Rifampicin in “pure” ethanol (1% chloroform), using the same spray drying conditions as described earlier in this Example, except the outlet temperature which was around 64° C. Formation of nanoparticles was not observed (FIG. 24B).

3) A solution of Rifampicin with lipids (60/40 w/w) in “pure” ethanol (1% chloroform), using the same spray drying conditions as described earlier in this Example, except the outlet temperature which was around 64° C. (FIG. 24C). Formation of nanoparticles was not observed.

It is reasonable to believe that the nanoparticles come from a co-precipitation of Rifampicin and the lipids, and that the mixture of the two solvents is necessary to obtain formation of these nanoparticles.

Formation of nanoparticles also occurred in other formulations such as DPPC-Sodium Citrate-Calcium Chloride when Rifampicin was added (see pictures below). Rifampicin was solubilized in 10 to 20 ml of chloroform and this solution was added to an ethanol solution containing DPPC (700 ml). The resulting solution was mixed with a water solution (300 ml) containing sodium citrate and/or calcium chloride just before spray drying. The solution contained 1 g of solutes: 60% Rifampicin (by weight) the rest being DPPC (between 28 and 40% by weight of solutes), sodium citrate (between 0 and 8% by weight of solutes) and calcium chloride (between 0 and 4% by weight of solutes).

Solutions were spray dried according to the following conditions: the inlet temperature was 110° C. and the outlet temperature approximately 45° C. The atomizer spin rate was 20000 RPM, using a V24 wheel. The liquid feed rate was 70 ml/min and the drying gas flow rate was around 98 kg/hr.

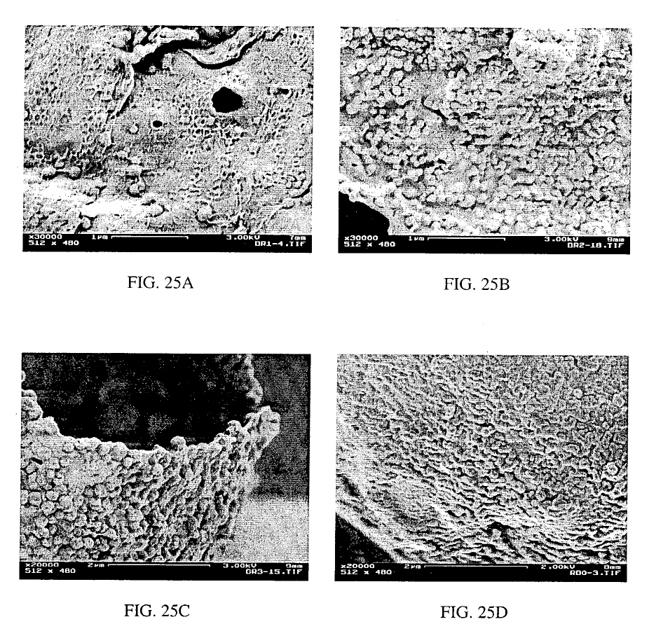

Nanoparticles in larger particles were always seen when Rifampicin was present with or without the salts (Sodium Citrate-Calcium Chloride) (FIGS. 25A-25D). Therefore, it is reasonable to believe that salts are not responsible for the formation of nanoparticles. It is noted however, that without salts, nanoparticles can take elongated shapes as well as spherical shapes.

While this invention has been particularly shown and described with references to preferred embodiments thereof, it will be understood by those skilled in the art that various changes in form and details may be made therein without departing from the scope of the invention encompassed by the appended claims.

What is claimed is:

1. A pharmaceutical composition comprising spray dried particles, said particles comprising sustained action nanoparticles, said nanoparticles comprising a bioactive agent and having a geometric diameter of about 1 micron or less.

2. The pharmaceutical composition of claim 1, wherein said nanoparticles have a geometric diameter of between about 25 nanometers and about 1 micron or less.

3. The pharmaceutical composition of claim 1, wherein said nanoparticles have a geometric diameter of between about 25 nanometers and less than 1 micron.

4. The pharmaceutical composition of claim 1, wherein said spray dried particles have an aerodynamic diameter between about 1 μm and about 6 μm.

5. The pharmaceutical composition of claim 1, wherein said spray dried particles comprises 100% by weight nanoparticles.

6. The pharmaceutical composition of claim 1, wherein said spray dried particles comprises at least 75% by weight nanoparticles.

7. The pharmaceutical composition of claim 1, wherein said spray dried particles comprises at least 50% by weight nanoparticles.

8. The pharmaceutical composition of claim 1, wherein said spray dried particles comprises at least 25% by weight nanoparticles.

9. The pharmaceutical composition of claim 1, wherein said spray dried particles comprises at least 5% by weight nanoparticles.

10. The pharmaceutical composition of claim 1, further comprising an additive.

11. The pharmaceutical composition of claim 10, wherein said additive is an excipient.

12. The pharmaceutical composition of claim 11, wherein said excipient is selected from the group consisting of phospholipids, polypeptides, polysaccharides, polyanhydrides, amino acids, polymers, proteins, surfactants, cholesterol, fatty acids, fatty acid esters, sugars and combinations thereof.

13. The pharmaceutical composition of claim 12, wherein said phospholipid is selected from the group consisting of phosphatidylcholines, phosphatidylethanolamines, phosphatidylglycerols, phosphatidylserines, phosphatidylinositols and combinations thereof.

14. The pharmaceutical composition of claim 10, wherein said additive is a bioactive agent.

15. The pharmaceutical composition of claim 14, wherein said bioactive agent is selected from the group consisting of a therapeutic agent, a diagnostic agent, and a prophylactic agent.

16. The pharmaceutical composition of claim 15, wherein said therapeutic agent is selected from the group consisting of insulin, estradiol, rifampin, ethambutol, pyrazinamide and albuterol.

17. The pharmaceutical composition of claim 10, wherein said additive is a second bioactive agent, and wherein the release of said second bioactive agent from said particles is faster than the release of said bioactive agent contained in said nanoparticle.

18. The pharmaceutical composition of claim 17, wherein said second bioactive agent and said bioactive agent comprising said nanoparticle are the same.

19. The pharmaceutical composition of claim 17, wherein said second bioactive agent and said bioactive agent comprising said nanoparticle are different.

20. The pharmaceutical composition of claim 17, wherein said additive is a second bioactive agent, and wherein the release of said second bioactive agent from said particles is a sustained release.

21. The pharmaceutical composition of claim 17, wherein said second bioactive agent is selected from the group consisting of a therapeutic agent, a diagnostic agent, and a prophylactic agent.

22. The pharmaceutical composition of claim 21, wherein said second bioactive agent is selected from the group consisting of insulin, estradiol, rifampin ethambutol and pyrazinamide.

23. The pharmaceutical composition of claim 1, wherein said nanoparticle is biodegradable.

24. The pharmaceutical composition of claim 23, wherein said nanoparticle is polymeric.

25. The pharmaceutical composition of claim 23, wherein said nanoparticle is nonpolymeric.

26. The pharmaceutical composition of claim 1, wherein said nanoparticle is non-biodegradable.

27. The pharmaceutical composition of claim 26, wherein said nanoparticle is polymeric.

28. The pharmaceutical composition of claim 27, wherein said nanoparticle comprises polystyrene.

29. The pharmaceutical composition of claim 28, further comprising lactose or hydroxypropylcellulose.

30. The pharmaceutical composition of claim 1, wherein said nanoparticle is a bead.

31. The pharmaceutical composition of claim 30, wherein said bead is a polystyrene bead.

32. The pharmaceutical composition of claim 30, wherein said bead is a polystyrene latex bead.

33. The pharmaceutical composition of claim 30, wherein said bioactive agent is incorporated into said bead.

34. The pharmaceutical composition of claim 1, wherein said composition is respirable.

35. The pharmaceutical composition of claim 1, wherein said particles are formulated to dissolve into said nanoparticles.

36. A pharmaceutical composition comprising phospholipid-containing biodegradable particles, said particles having a geometric diameter of between about 4 microns and about 8 microns and an aerodynamic diameter of between about 1 micron and about 3 microns, said particles comprising between about 5% and about 80% by weight nanoparticles, said nanoparticles having a geometric diameter of between about 25 nanometers and about 1 micron, and wherein said nanoparticles are carboxylate modified polystyrene beads.

37. A pharmaceutical composition comprising phospholipid-containing biodegradable particles, said particles having a geometric diameter of between about 5 microns and about 8 microns and an aerodynamic diameter of between about 2.5 and about 3.5, said particles comprising between about 5% and about 70% by weight nanoparticles, said nanoparticles having a geometric diameter of between about 25 nanometers and about 1 micron, and wherein said nanoparticles are carboxylate modified polystyrene beads.

38. A pharmaceutical composition comprising phospholipid-containing biodegradable particles, said particles having a geometric diameter of between about 8 microns and about 12.5 microns and an aerodynamic diameter of between about 2 microns and about 3 microns, said particles comprising between about 5 and about 85% by weight nanoparticles, said nanoparticles having a geometric diameter of between about 25 nanometers and about 1 micron, and wherein said nanoparticles are carboxylate modified polystyrene beads.

39. A pharmaceutical composition comprising phospholipid-containing biodegradable particles, said particles having a geometric diameter of between about 7.5 microns and about 15 microns and an aerodynamic diameter of between about 4.5 and about 7.5, said particles comprising between 5 and 90% by weight nanoparticles, said nanoparticles having a geometric diameter of between about 25 nanometers and about 1 micron, and wherein said nanoparticles are colloidal silica.

40. A pharmaceutical composition comprising phospholipid-containing biodegradable particles and nanoparticles, wherein said nanoparticles comprise Rifampicin and one or more phospholipids.

41. A method of treating a condition in a patient, comprising the step of administering to said patient a pharmaceutical composition comprising spray dried particles, said particles comprising sustained action nanoparticles, said nanoparticles comprising a bioactive agent and having a geometric diameter of about 1 micron or less.

42. The method of claim 41, wherein said nanoparticles have a geometric diameter of between about 25 nanometers and less than 1 micron.

43. The method of claim 41, wherein said spray dried particles have an aerodynamic diameter between about 1 micron and about 10 microns.

44. The method of claim 41, wherein said spray dried particles comprise 100% by weight nanoparticles.

45. The method of claim 41, wherein said spray dried particles comprise at least 75% by weight nanoparticles.

46. The method of claim 41, wherein said spray dried particles comprise at least 50% by weight nanoparticles.

47. The method of claim 41, wherein said spray dried particles comprise at least 25% by weight nanoparticles.

48. The method of claim 41, wherein said spray dried particles comprise at least 5% by weight nanoparticles.

49. The method of claim 41, wherein said pharmaceutical composition further comprises an additive.

50. The method of claim 49, wherein said additive is an excipient.

51. The method of claim 50, wherein said excipient is selected from the group consisting of phospholipids, polypeptides, polysaccharides, polyanhydrides, amino acids, polymers, proteins, surfactants, cholesterol, fatty acids, fatty acid esters, sugars and combinations thereof.

52. The method of claim 51, wherein said phospholipid is selected from the group consisting of phosphatidylcholines, phosphatidylethanolamines, phosphatidylglycerols, phosphatidylserines, phosphatidylinositols and combinations thereof.

53. The method of claim 49, wherein said additive is a bioactive agent.

54. The method of claim 53, wherein said bioactive agent is selected from the group consisting of a therapeutic agent, a diagnostic agent, and a prophylactic agent.

55. The method of claim 54, wherein said therapeutic agent is selected from the group consisting of insulin, estradiol, rifampin, ethambutol, pyrazinamide and albuterol.

56. The method of claim 49, wherein said additive is a second bioactive agent, and wherein the release of said second bioactive agent from said particles is faster than the release of said bioactive agent contained in said nanoparticle.

57. The method of claim 56, wherein said second bioactive agent and said bioactive agent comprising said nanoparticle are the same.

58. The method of claim 56, wherein said second bioactive agent and said bioactive agent comprising said nanoparticle are different.

59. The method of claim 56, wherein said additive is a second bioactive agent, and wherein the release of said second bioactive agent from said particles is a sustained release.

60. The method of claim 56, wherein said second bioactive agent is selected from the group consisting of a therapeutic agent, a diagnostic agent, and a prophylactic agent.

61. The method of claim 60, wherein said second bioactive agent is selected from the group consisting of insulin, estradiol, rifampin, ethambutol and pyrazinamide.

62. The method of claim 41, wherein said nanoparticle is biodegradable.

63. The method of claim 62, wherein said nanoparticle is polymeric.

64. The method of claim 62, wherein said nanoparticle is nonpolymeric.

65. The method of claim 41, wherein said nanoparticle is non-biodegradable.

66. The method of claim 65, wherein said nanoparticle is polymeric.

67. The method of claim 66, wherein said nanoparticle comprises polystyrene.

68. The method of claim 65, wherein said nanoparticle is nonpolymeric.

69. The method of claim 41, wherein said nanoparticle is a bead.

70. The method of claim 69, wherein said bead is a polystyrene bead.

71. The method of claim 69, wherein said bead is a polystyrene latex bead.

72. The method of claim 69, wherein said bioactive agent is incorporated into said bead.

73. The method of claim 41, wherein said pharmaceutical composition is respirable.

74. The method of claim 73, wherein said administering is done by inhalation.

75. The method of claim 74, wherein said inhalation comprises delivery primarily to the deep lung.

76. The method of claim 74, wherein said inhalation comprises delivery primarily to the central airways.

77. The method of claim 74, wherein said inhalation comprises delivery primarily to the upper airways.

78. The method of claim 41, wherein said particles are formulated to release said nanoparticles.

79. A method of making spray dried particles comprising sustained action nanoparticles, said nanoparticles comprising a bioactive agent and having a geometric diameter of about 1 micron or less, said method comprising the steps of spray drying a solution comprising said nanoparticles or reagents capable of forming nanoparticles under conditions that form spray dried particles.

80. The method of claim 79, wherein said nanoparticles have a geometric diameter of between about 25 nanometers and less than 1 micron.

81. The method of claim 79, wherein said spray dried particles have an aerodynamic diameter between about 1 micron and about 13 microns.

82. The method of claim 79, wherein said spray dried particles comprises at least 100% by weight nanoparticles.

83. The method of claim 79, wherein said spray dried particles comprises at least 75% by weight nanoparticles.

84. The method of claim 79, wherein said spray dried particles comprises at least 50% by weight nanoparticles.

85. The method of claim 79, wherein said spray dried particles comprises at least 25% by weight nanoparticles.

86. The method of claim 79, wherein said spray dried particles comprises at least 5% by weight nanoparticles.

87. The method of claim 79, wherein said spray dried particles further comprises an additive.

88. The method of claim 87, wherein said additive is an excipient.

89. The method of claim 88, wherein said excipient is selected from the group consisting of phospholipids, polypeptides, polysaccharides, polyanhydrides, amino acids, polymers, proteins, surfactants, cholesterol, fatty acids, fatty acid esters, sugars and combinations thereof.

90. The method of claim 89, wherein said phospholipid is selected from the group consisting of phosphatidylcholines, phosphatidylethanolamines, phosphatidylglycerols, phosphatidylserines, phosphatidylinositols and combinations thereof.

91. The method of claim 87, wherein said additive is a bioactive agent.

92. The method of claim 91, wherein said bioactive agent is selected from the group consisting of a therapeutic agent, a diagnostic agent, and a prophylactic agent.

93. The method of claim 92, wherein said therapeutic agent is selected from the group consisting of insulin, estradiol, rifampin, ethambutol, pyrazinamide and albuterol.

94. The method of claim 87, wherein said additive is a second bioactive agent, and wherein the release of said second bioactive agent from said particles is faster than the release of said bioactive agent contained in said nanoparticle.

95. The method of claim 94, wherein said second bioactive agent and said bioactive agent comprising said nanoparticle are the same.

96. The method of claim 94, wherein said second bioactive agent and said bioactive agent comprising said nanoparticle are different.

97. The method of claim 94, wherein said additive is a second bioactive agent, and wherein the release of said second bioactive agent from said particles is a sustained release.

98. The method of claim 94, wherein said second bioactive agent is selected from the group consisting of a therapeutic agent, a diagnostic agent, and a prophylactic agent.

99. The method of claim 98, wherein said second bioactive agent is selected from the group consisting of insulin, estradiol, rifampin, ethambutol and pyrazinamide.

100. The method of claim 79, wherein said nanoparticle is biodegradable.

101. The method of claim 100, wherein said nanoparticle is polymeric.

102. The method of claim 100, wherein said nanoparticle is nonpolymeric.

103. The method of claim 79, wherein said nanoparticle is non-biodegradable.

104. The method of claim 103, wherein said nanoparticle is polymeric.

105. The method of claim 104, wherein said nanoparticle comprises polystyrene.

106. The method of claim 103, wherein said nanoparticle is nonpolymeric.

107. The method of claim 79, wherein said nanoparticle is a bead.

108. The method of claim 107, wherein said bead is a polystyrene bead.

109. The method of claim 107, wherein said bead is a polystyrene latex bead.

110. The method of claim 107, wherein said bioactive agent is incorporated into said bead.

111. The method of claim 79, wherein said pharmaceutical composition is respirable.

112. The method of claim 79, wherein said particles are formulated to dissolve into said nanoparticles.

113. The method of claim 41, wherein said nanoparticles have a geometric diameter of between about 25 nanometers and about 1 micron or less.

114. The method of claim 79, wherein said nanoparticles have a geometric diameter of between about 25 nanometers and about 1 micron or less.

115. A composition comprising spray dried particles, said particles comprising sustained action nanoparticles, said nanoparticles comprising a nutraceutical agent and having a geometric diameter of about 1 micron or less.

116. The composition of claim 115, wherein said nanoparticles have a geometric diameter of between about 25 nanometers and about 1 micron or less.

117. The composition of claim 115, wherein said nanoparticles have a geometric diameter of between about 25 nanometers and less than 1 micron.

118. The composition of claim 115, wherein said spray dried particles have an aerodynamic diameter between about 1 μm and about 6 μm.

119. The composition of claim 115, wherein said spray dried particles comprises 100% by weight nanoparticles.

120. The composition of claim 115, wherein said spray dried particles comprises at least 75% by weight nanoparticles.

121. The composition of claim 115, wherein said spray dried particles comprises at least 50% by weight nanoparticles.

122. The composition of claim 115, wherein said spray dried particles comprises at least 25% by weight nanoparticles.

123. The composition of claim 115, wherein said spray dried particles comprises at least 5% by weight nanoparticles.

124. A method of treating a nutritional deficiency in a patient comprising the step of administering to said patient a composition comprising spray dried particles, said particles comprising sustained action nanoparticles, said nanoparticles comprising a nutraceutical agent and having a geometric diameter of about 1 micron or less.

125. The method of claim 124, wherein the nutraceutical agent is selected from the group consisting of a vitamin, a mineral and a nutritional supplement.
